(19) United States
(12) Patent Application Publication
(10) Pub. No.: US 2023/0271002 A1
(43) Pub. Date: Aug. 31, 2023

(54) HYDROGEL IONIC CIRCUIT BASED DEVICES FOR ELECTRICAL STIMULATION AND DRUG THERAPY

(71) Applicant: Board of Regents of the University of Nebraska, Lincoln, NE (US)

(72) Inventors: Siwei ZHAO, OMAHA, NE (US); Fan ZHAO, OMAHA, NE (US)

(73) Assignee: Board of Regents of the University of Nebraska, Lincoln, NE (US)

(21) Appl. No.: 18/006,116

(22) PCT Filed: Jul. 29, 2021
(86) PCT No.: PCT/US2021/043718

§ 371 Date: Jan. 19, 2023

Related U.S. Application Data
(60) Provisional application No. 63/059,490, filed on Jul. 31, 2020.

(51) Int. Cl. A61N 1/30; A61N 1/04

(52) U.S. Cl. CPC A61N 1/303; A61N 1/0444

(57) ABSTRACT

A hydrogel ionic circuit (HIC) electrode configured for electrical stimulation and/or drug therapy (e.g., iontophoresis) is disclosed. The HIC electrode includes a chamber containing a salt solution. The chamber is at least partially bound by a hydrogel membrane that defines a barrier for the salt solution. The HIC electrode further includes an electrode configured to apply an electrical current to the chamber to induce an ion current in the salt solution, wherein the hydrogel membrane is ionically conductive and configured to transmit the ion current.

### CROSS-REFERENCE TO RELATED APPLICATIONS

The present application claims priority to U.S. Provisional Application No. 63/059,490, filed Jul. 31, 2020 and titled “Hydrogel Ionic Circuit for Electrical Stimulation and Drug Therapy,” which is incorporated herein by reference in its entirety.

### TECHNICAL FIELD

The present disclosure relates to devices that employ an electrical current for therapeutic applications.

### BACKGROUND

There are a number of therapeutic applications that employ an electrical current. Examples include electrical stimulation and iontophoresis. However, the existing bioelectronic systems have not fully resolved mismatches between engineered circuits and biological systems. Consequently, electrical current-based therapies often result in pain and/or damage to biological tissues.

In order to avoid irreversible damage to biological tissues, some electrical current-based therapies can only be applied at low current intensity and may be limited in this regard. For instance, iontophoresis applied at low current intensity is inefficient at transporting macromolecule drugs and nanoparticles due to their larger sizes.

To address limitations of existing bioelectronic systems, there is a need for improved devices that can employ high current intensity while maintaining acceptable levels of pH and thermal energy at the device-to-biological tissue interface to prevent pain and/or damage to biological tissues.

### SUMMARY

A hydrogel ionic circuit (HIC) electrode configured for electrical stimulation and/or drug therapy (e.g., iontophoresis) is disclosed. In embodiments, the HIC electrode includes a chamber containing a salt solution. The chamber is at least partially bound by a hydrogel membrane that defines a barrier for the salt solution. The HIC electrode further includes an electrode configured to apply an electrical current to the chamber to induce an ion current in the salt solution, wherein the hydrogel membrane is ionically conductive and configured to transmit the ion current.

In a sense, the HIC electrode is a buffered electrode that mitigates thermal and/or pH changes at a device-to-biological tissue interface for therapeutic applications that employ an electrical current. The HIC electrode converts the electrical current to ion current that can be transmitted through the hydrogel membrane. This may allow for the use of electrical current at higher current intensity than would otherwise be possible for electrical stimulation, iontophoresis, and other therapeutic applications that employ an electrical current.

An iontophoresis device that incorporates at least one HIC electrode is also disclosed. In embodiments, the iontophoresis device includes a first chamber containing a salt solution and a second chamber containing a therapeutic solution, wherein the second chamber is configured to interface with a portion of a surface overlaying a target region. The iontophoresis device further includes a hydrogel membrane separating the first chamber from the second chamber. An electrode is configured to apply an electrical current to the first chamber to induce an ion current in the salt solution, wherein the ion current acts on the second chamber to iontophoretically transport molecules (e.g., drug molecules) from the therapeutic solution across the surface to the target region.

An electrical stimulation device for wound healing that incorporates at least one HIC electrode is also disclosed. In embodiments, the electrical stimulation device includes a substrate configured to overlay a cutaneous wound. The substrate may have a plurality of channels embedded within or attached to the substrate, with each of the channels containing a salt solution and being at least partially bound by a hydrogel membrane that defines a barrier between the salt solution and the cutaneous wound. An electrode is configured to apply an electrical current to a channel of the plurality of channels to induce an ion current in the salt solution, wherein the ion current acts on the cutaneous wound to stimulate healing.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is a schematic illustration of a hydrogel ionic circuit (HIC), in accordance with an example embodiment of the present disclosure.

FIG. 2A is a schematic illustration of an iontophoresis system, in accordance with an example embodiment of the present disclosure.

FIG. 2B is a schematic illustration of a HIC electrode for iontophoresis, in accordance with an example embodiment of the present disclosure.

FIG. 2C is a schematic illustration of a typical electrode for iontophoresis, in accordance with an example embodiment of the present disclosure.

FIG. 2D is a schematic illustration of a HIC-based iontophoresis system, in accordance with an example embodiment of the present disclosure.

FIG. 3 is a schematic illustration of a HIC-based iontophoresis device, in accordance with an example embodiment of the present disclosure.

FIG. 4 is a schematic illustration of a HIC-based iontophoresis system for performing ex vivo testing with a HIC-based iontophoresis device, in accordance with an example embodiment of the present disclosure.

FIG. 5A is a chart illustrating phase separation behaviors of polyethylene glycol (PEG) hydrogels after soaking in anode and cathode salt solution overnight, respectively, in accordance with an example embodiment of the present disclosure.

FIG. 5B is a chart illustrating long-term stability of PEG hydrogel conductivity after soaking in salt solution for two weeks, in accordance with an example embodiment of the present disclosure.

FIG. 5C is a chart illustrating conductivity changes of high-concentration salt solutions and buffer solutions after a HIC device is immersed for different periods of time, in accordance with an example embodiment of the present disclosure.

FIG. 5D is also a chart illustrating conductivity changes of high-concentration salt solutions and buffer solutions after a HIC device is immersed for different periods of time, in accordance with an example embodiment of the present disclosure.

FIG. 5E is a chart illustrating pH changes of high-concentration salt solutions and buffer solutions after HIC electrodes are immersed for different periods of time, in accordance with an example embodiment of the present disclosure.

FIG. 5F is also a chart illustrating pH changes of high-concentration salt solutions and buffer solutions after HIC electrodes are immersed for different periods of time, in accordance with an example embodiment of the present disclosure.

FIG. 5G illustrates cell viability results for four types of ocular cells after HIC electrodes were immersed in cell culture media for 1 hour, in accordance with an example embodiment of the present disclosure.

FIG. 6A is a schematic illustration of a HIC-based iontophoresis system for performing ex vivo testing with a HIC-based iontophoresis device, in accordance with an example embodiment of the present disclosure.

FIG. 6B is a chart illustrating temperature changes of ocular surface and vitreous during iontophoresis at 100 mA for 15 minutes, in accordance with an example embodiment of the present disclosure.

FIG. 6C is a chart illustrating highest temperatures of current applied to a surface of an eyeball during iontophoresis at 100 mA for 15 minutes with HIC electrodes and carbon electrodes, in accordance with an example embodiment of the present disclosure.

FIG. 6D is a chart illustrating pH changes of buffer solution filled in drug donor after iontophoresis at 100 mA for 15 minutes with HIC electrodes and carbon electrodes, in accordance with an example embodiment of the present disclosure.

FIG. 6E is a chart illustrating pH changes of current applied to a surface of an eyeball after iontophoresis at 100 mA for 15 minutes with HIC electrodes and carbon electrodes, in accordance with an example embodiment of the present disclosure.

FIG. 6F is a chart illustrating pH changes of vitreous fluid isolated from eyeballs after electrical stimulation at 100 mA for 15 minutes with HIC electrodes and carbon electrodes, in accordance with an example embodiment of the present disclosure.

FIG. 6G depicts porcine eyeballs after ex vivo iontophoresis tests performed with HIC electrodes and carbon electrodes, in accordance with an example embodiment of the present disclosure.

FIG. 7A is a schematic illustration of a Franz diffusion cell test system for performing ex vivo testing of transscleral drug delivery with a HIC-based iontophoresis device, in accordance with an example embodiment of the present disclosure.

FIG. 7B is a chart illustrating an accumulated amount of FD-40 collected from the receipt chamber of the Franz diffusion cell test system when applying different current, in accordance with an example embodiment of the present disclosure.

FIG. 7C is a chart illustrating the permeability coefficient of FD-40 through porcine sclera as a function of applied current, in accordance with an example embodiment of the present disclosure.

FIG. 7D is a chart illustrating the FD-40 enhancement factor as a function of current, in accordance with an example embodiment of the present disclosure.

FIG. 7E is a chart illustrating an accumulated amount of FD-40 collected from the receipt chamber of the Franz diffusion cell test system when applying the same charge with different current, in accordance with an example embodiment of the present disclosure.

FIG. 7F is a chart illustrating an accumulated amount of FITC-dextran delivered through the porcine sclera using FITC-dextran with different molecular sizes under 100 mA iontophoresis for 15 minutes, in accordance with an example embodiment of the present disclosure.

FIG. 7G is a chart illustrating an accumulated amount of FD-40 delivered through the porcine sclera with different concentrations under 100 mA iontophoresis for 15 minutes, in accordance with an example embodiment of the present disclosure.

FIG. 7H depicts a representative fluorescein image of the cryo-sectioned porcine sclera after transscleral iontophoresis test under passive diffusion for 15 minutes, in accordance with an example embodiment of the present disclosure.

FIG. 7I depicts a representative fluorescein image of the cryo-sectioned porcine sclera after transscleral iontophoresis test under conventional iontophoresis conditions (4.5 mA (7.5 mA/cm2)) for 15 minutes, in accordance with an example embodiment of the present disclosure.

FIG. 7J depicts a representative fluorescein image of the cryo-sectioned porcine sclera after transscleral iontophoresis test under high ionic current (100 mA (157 mA/cm2)) for 15 minutes, in accordance with an example embodiment of the present disclosure.

FIG. 8A is a chart illustrating an accumulated amount of FD-40 collected from the vitreous of an excised rabbit eyeball after iontophoresis at 100 mA for 20 minutes using a HIC-based iontophoresis device, in accordance with an example embodiment of the present disclosure.

FIG. 8B is a chart illustrating an accumulated amount of FD-40 in various posterior segments of an excised rabbit eyeball after iontophoresis at 100 mA for 20 minutes using a HIC-based iontophoresis device, and after iontophoresis at 100 mA for 20 minutes with post-iontophoretic passive diffusion for 21 hours, in accordance with an example embodiment of the present disclosure.

FIG. 8C is a chart illustrating an accumulated amount of Avastin in the vitreous humor of an excised rabbit eyeball after iontophoresis at 100 mA for 20 minutes using a HIC-based iontophoresis device, in accordance with an example embodiment of the present disclosure.

FIG. 8D is a chart illustrating an accumulated amount of Bevacizumab collected from the vitreous of an excised rabbit eyeball after iontophoresis at 100 mA for 20 minutes using a HIC-based iontophoresis device, in accordance with an example embodiment of the present disclosure.

FIG. 8E illustrates retinal pigmented epithelium cell and choroid/retina endothelial cell viability when conducting high ionic current test conditions using a HIC-based iontophoresis device, in accordance with an example embodiment of the present disclosure.

FIG. 8F depicts H&E images for histological analysis of the sclera after iontophoresis on freshly excised rabbit eyeballs under different test conditions (no treatment, conventional iontophoresis condition (10 mA for 20 min, Cl), 100 mA for 20 min using HIC-based iontophoretic device, and 100 mA for 20 min using carbon electrodes), in accordance with an example embodiment of the present disclosure.

FIG. 9A depicts fluorescein images of a cryo-sectioned rabbit cornea, wherein the fluorescent intensity was converted to a 3D stack using surface plotting software, in accordance with an example embodiment of the present disclosure.

FIG. 9B is a chart illustrating an accumulated amount of FD-40 extracted from cornea under different test conditions using a HIC-based iontophoresis device, in accordance with an example embodiment of the present disclosure.

FIG. 9C is a chart illustrating an accumulated amount of bevacizumab (an anti-VEGF) extracted from cornea after iontophoresis at 100 mA for 10 minutes using a HIC-based iontophoresis device loaded with a drug solution including 10 mg/mL Avastin, in accordance with an example embodiment of the present disclosure.

FIG. 9D illustrates corneal epithelial and endothelial cell viability when conducting high ionic current test conditions using a HIC-based iontophoresis device, in accordance with an example embodiment of the present disclosure.

FIG. 9E depicts H&E images for histological analysis of the cornea after iontophoresis on freshly excised rabbit eyeballs under different test conditions, in accordance with an example embodiment of the present disclosure.

FIG. 10A is a chart illustrating average size and polydispersity index of bare and chitosan coated FITC-PLGA nanoparticles, in accordance with an example embodiment of the present disclosure.

FIG. 10B is a chart illustrating surface charge of bare and chitosan coated FITC-PLGA nanoparticles, in accordance with an example embodiment of the present disclosure.

FIG. 10C is a chart illustrating results of passive diffusion, conventional iontophoresis, and HIC-based iontophoresis with PLGA nanoparticles, in accordance with an example embodiment of the present disclosure.

FIG. 10D is also a chart illustrating results of passive diffusion, conventional iontophoresis, and HIC-based iontophoresis with PLGA nanoparticles, in accordance with an example embodiment of the present disclosure.

FIG. 11A is a schematic cross-sectional side view of a HIC-based iontophoresis device embedded within an ocular lens, in accordance with an example embodiment of the present disclosure.

FIG. 11B is a schematic top plan view of a HIC-based iontophoresis device embedded within an ocular lens, in accordance with an example embodiment of the present disclosure.

FIG. 12A is a schematic top plan view of a HIC-based electrical stimulation device, in accordance with an example embodiment of the present disclosure.

FIG. 12B is a schematic cross-sectional side view of a HIC-based electrical stimulation device, in accordance with an example embodiment of the present disclosure.

FIG. 12C is a schematic cross-sectional side view of a HIC-based electrical stimulation device with a splint configured to hold the electrical stimulation device against a cutaneous wound, in accordance with an example embodiment of the present disclosure.

### DETAILED DESCRIPTION

Electrical stimulation is a non-invasive and non-pharmacological physical stimulus. Electrical stimulation has a broad range of biomedical effects. At the molecular level, it can facilitate the transport of both charged and uncharged biomolecules through biological membranes via electrophoresis and electroosmosis. These two processes collectively are called iontophoresis. At the cellular level, electrical stimulation can interact with a variety of cellular components, such as ion channels, membrane-bound proteins, cytoskeleton, and intracellular organelles. These interactions alter cellular activities and functions, such as contraction, migration, orientation, and proliferation. Electrical stimulation shows strong clinical potential for drug delivery, tissue regeneration, and wound healing. However, the existing bioelectronic systems have not fully resolved mismatches between engineered circuits and biological systems, resulting in pain and/or damage to biological tissues.

In this disclosure, aqueous two-phase systems are utilized to generate programmable hydrogel ionic circuits (HICs). High-conductivity salt-solution patterns are stably encapsulated within hydrogel matrices using salt phase separation, which route ionic current with high resolution and enable localized delivery of electrical stimulation. This strategy allows designer electronics that match biological systems, including transparency, complete aqueous-based connective interface, distribution of ionic electrical signals between engineered and biological systems, and avoidance of tissue damage from electrical stimulation.

The library of conductive materials used in currently available bioelectronic devices typically includes metals, carbon-based materials, and conductive polymers. Such requirements significantly increase both design complexity and footprint, and fundamentally affect the conductivity of the device. Moreover, most existing conductive materials exhibit a mechanical mismatch with human tissues, making them unsuitable for long-term wear and implantable applications. Critically, the conductive materials used in the devices carry electron (or, in some cases, hole) current, which has to be converted to ion current at the electrode/electrolyte interfaces through electrochemical reactions in order to stimulate the biological systems. This is particularly important for voltage drops across the interfaces higher than a certain threshold (typically 1 V for water), which induces local heat (through Joule heating), pH changes, electrode degradation, and the generation of highly reactive chemical species. These reactions can cause pain and damage to the biological tissues, an issue especially relevant for the long-term or high current electrostimulation, such as iontophoresis. Thus, new options for materials and device designs are needed to facilitate a new generation of biocompatible electronic systems that can interface with living systems.

Ionically conductive hydrogel materials have been developed that offer intrinsic biocompatibility, a mechanical match to tissues, and can potentially be engineered to possess degradability. These hydrogel conductors utilize ionic charge transport, thus eliminating electron-to-ionic current conversion at biological interfaces (and the associated adverse effects) and enable seamless and safe interfaces with the biological tissues. Specifically, the ionic hydrogel conductors allow the electron-conducting materials (e.g., metal electrodes) to be separated from the biological tissues; chemical changes at electron-conductor/hydrogel interfaces induced by the electrochemical reactions can be sufficiently buffered before reaching biological tissues. Moreover, due to the high water content of hydrogels, the heat generated by current injection dissipates rather than accumulating on the tissue surface, reducing local burns and pain typically caused by traditional conductors.

The ionic hydrogel conductors (e.g., polyethylene glycol, dextran, dipotassium phosphate, ethanol, etc.) have been utilized as electrodes in electromechanical systems and as interconnects for constructing hydrogel circuits on insulating silicone substrates. However, the existing ionic hydrogel conductors either cannot form stable interconnect patterns in aqueous environments due to ion diffusion or possess low conductivity, which hinder the expansion of their applicability to the integrated electronic systems in biologically relevant environments. This disclosure presents complex, aqueous-stable, HICs enabled by the salt aqueous two-phase systems and compatible for direct interfaces with the living systems. Furthermore, the hydrogel ionic design of ionically conductive patterns can be mechanically reprogrammed and modulated after fabrication. Additional utility of these HICs is demonstrated by delivering localized electrical stimulation in the biological environments with reduced adverse effects when compared to the conventional metal- and carbon-based electrodes.

Various embodiments of this disclosure are directed to a HIC-based device for ocular drug delivery and electrical stimulation iontophoresis. The disclosed device serves as an alternative to intraocular injections for posterior segment drug delivery which causes changes in ocular pressure capable of causing adverse effects including retinal detachment, endophthalmitis, hemorrhages, and rise in intraocular pressure.

Macromolecular and nanoparticle (NP) ophthalmic drugs have seen increasing utility in ocular disease treatment. Macromolecules, primarily monoclonal antibodies against human vascular endothelial growth factor (anti-VEGF), have been successfully used in treating a wide range of eye conditions, including neovascular age-related macular degeneration (AMD), diabetic macular edema, proliferative diabetic retinopathy, corneal neovascularization, and neovascular glaucoma. NP formula provides better solubility for hydrophobic drugs, sustained release over a prolonged period of time, and the ability to target specific tissues through surface modification. Several NP ophthalmic drugs have been FDA-approved to treat dry eye syndrome (cyclosporine nanoemulsion) and for photodynamic therapy (verteporfin liposome). More clinical trials are underway to test NP ophthalmic drugs in the treatment of macular degeneration, cataracts, glaucoma, ocular infection, and hypertension.

Despite the promising therapeutic efficacy of macromolecular and NP ophthalmic drugs, their intraocular delivery presents a significant challenge. The large size of macromolecular and NP drugs leads to a slower permeation rate through ocular tissue barriers compared to small molecule drugs. When eye drops are used, only a small fraction (typically less than 1%) of the applied macromolecule and NP drugs is delivered into the eye even with multiple doses per day. Although drug-eluting contact lenses effectively increase the residence time of drugs and are capable of sustained drug release, they do not enhance the drug permeation rate in ocular tissues. As a result, drug-eluting contact lenses typically need to be worn for an extended period of time (several days to weeks) to provide sustained therapeutically effective drug concentration. Extended wear of contact lens can cause irritation to ocular tissues and discomfort due to friction, which have shown to adversely affect the public acceptance of contact lenses. An anti-VEGF (ranibizumab)-eluting contact lens has been developed, but it failed to deliver a therapeutically efficacious concentration of ranibizumab into posterior segment tissue despite extended wear for several days. Systemically administered macromolecules and NPs need to overcome the blood-aqueous and blood-retinal barriers, and are subject to liver modification and kidney clearance. These lead to a very low bioavailability of typically less than 0.1%. Due to the challenges of topical and systemic routes, intraocular injection remains the most effective method for delivering macromolecular and NP ophthalmic drugs. However, the invasive injection procedure carries a risk of potentially blinding complications, including retinal pigment epithelium tear, retinal detachment, and endophthalmitis. Needle phobia can cause anxiety in patients, which negatively affects patient adherence to the treatment. Moreover, intraocular injection needs to be performed by a specialist. The uneven distribution of specialists between rural and urban areas and between developing and developed countries leads to a disparate treatment provision. There is a critical need for a next generation intraocular macromolecular and NP drug delivery technology that is safe, non-invasive, highly efficacious, and allows easy operation by patients or their caregivers without special trainings.

Iontophoresis is a constant (DC) electrical current-based, non-invasive drug delivery technology. It can be used to deliver both charged and neutral drug molecules and can be easily applied using portable or wearable devices. The potential of iontophoresis in ocular drug delivery has been explored, and a variety of different drugs from small molecules (e.g., steroids, oligonucleotides, antibiotics, and riboflavin) to macromolecules (e.g., anti-VEGF and nanoparticles have been tested. Although effective in delivering small-molecule drugs, current ocular iontophoresis has low efficiency when delivering macromolecule drugs and nanoparticles due to their larger sizes. Transscleral iontophoresis of Avastin (bevacizumab, M.W. 148 kDa) has been previously studied using isolated human sclera in vitro. When 3.8 mA/cm2 anodal iontophoresis was applied for up to 2 h, the permeation was only enhanced by 7.5 compared to passive diffusion. Anodal iontophoretic (1.8 mA/cm2 for 20 min) delivery of Avastin has been previously studied in vivo using the Visulex iontophoresis system. The total amount of Avastin delivered into the eye was 353±42 μg. Only 5±3 μg was observed in the retina/choroid tissues, without data reported in vitreous humor, which is not effective since at least 600 μg was delivered in the vitreous humor to treat wet AMD by intravitreal injection. Previous studies have performed 6 mA/cm2 iontophoresis of charged nanoparticles (20-45 nm) for 5 min on rabbit cornea and sclera in vivo, while the improvement of nanoparticle permeation in ocular tissues is still limited. In order to enhance the delivery efficiency of macromolecules and nanoparticles, higher current intensity is required, since iontophoretic efficiency is proportional to the current intensity applied. However, applying higher current intensities presents significant challenge for current ocular iontophoresis devices. All current devices employ electrodes that conduct electron currents. These electron currents have to be converted to ion currents at the electrode/tissue interface, because biological tissues conduct ion currents. For DC current, this conversion requires electrochemical (EC) reactions. These EC reactions can induce significant pH change (due to water electrolysis) and local heating (due to electrode overpotential) that can damage tissues when the current intensity is high. Previous studies have reported that ocular iontophoresis is considered safe when current intensity does not exceed 5.5 mA/cm2, and the duration does not exceed 40 minutes. Studies have reported that significant ocular damage can be induced at 15 mA/cm2.

A potential solution to this issue is the newly developed ionically conductive materials. They conduct ion currents, so when they are used as electrodes, EC reaction-based current conversion does not happen at the electrode/tissue interface. These materials include hydrogels containing high-concentration NaCl/LiCl, ionic liquid hydrogels, natural and synthesized polyelectrolyte hydrogels. However, NaCl/LiCl-containing hydrogels are not stable in aqueous tissue environments due to ion diffusion. Polyelectrolytes and ionic liquids have low conductivity (in general less than 2 S/m), which dissipates/attenuates electrical energy and increases Joule heating. Ionic liquids also have yet to address the issue of cyto- and tissue-toxicity. Therefore, these materials are not suitable for ocular iontophoresis at high current intensities.

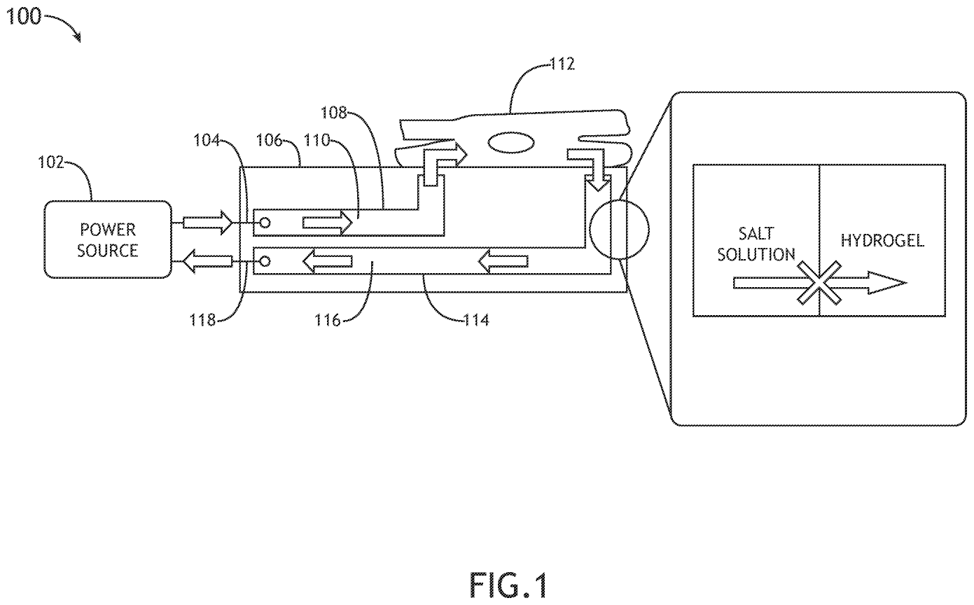

To address the issues of current ionic conductors, a novel ion current-conducting circuit device has been developed, referred to herein as the “hydrogel ionic circuit (HIC)”. FIG. 1 is a schematic illustration of a HIC 100, in accordance with an example embodiment of the present disclosure. The HIC 100 includes channels 108 and 114 filled with salt solutions 110 and 116, respectively, (e.g., saturated phosphate, sulfate, citrate, carbonate, or polyacrylate salt solution (up to 10.6 S/m in some embodiments)). The HIC channels 108 and 114 are encapsulated in a hydrogel membrane 106 (e.g., a polyethylene glycol (PEG) hydrogel matrix), thereby enabling the salt ions to be stably contained in the channels 108 and 114 due to aqueous two-phase separation (ATPS). The HIC 100 can route high ionic current and reduce the adverse effects associated with electrical stimulation at the HIC/tissue interface. For example, in the embodiment illustrated in FIG. 1, a power source 102 may be coupled to an electrode 104 and a counter electrode 118 (to complete the circuit). The electrode 104 may be configured to apply an electrical current to channel 108 to induce an ion current in salt solution 110, wherein the hydrogel membrane 106 is ionically conductive and configured to transmit the ion current to biological tissue 112. The counter electrode 118 may be coupled to channel 114 so that the ion current flows from channel 108 through a portion of the biological tissue 112 to channel 114. The combination of each metal/carbon electrode 104/118 and its corresponding HIC channel 108/114 acts as a buffered electrode that mitigates thermal and/or pH changes at a device-to-biological tissue interface for therapeutic applications that employ an electrical current. The HIC 100 converts the electrical current to ion current that can be transmitted through the hydrogel membrane 106 to the biological tissue 112. This may allow for the use of electrical current at higher current intensity than would otherwise be possible for electrical stimulation, iontophoresis, and other therapeutic applications that employ an electrical current.

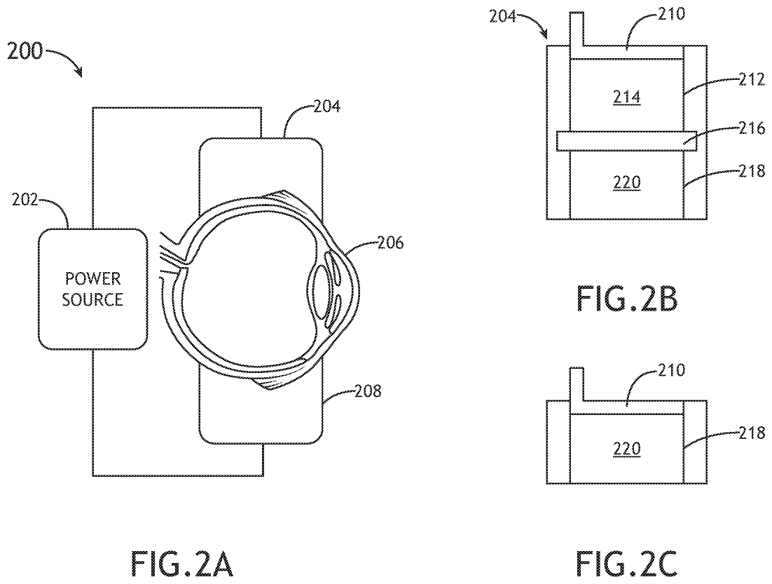

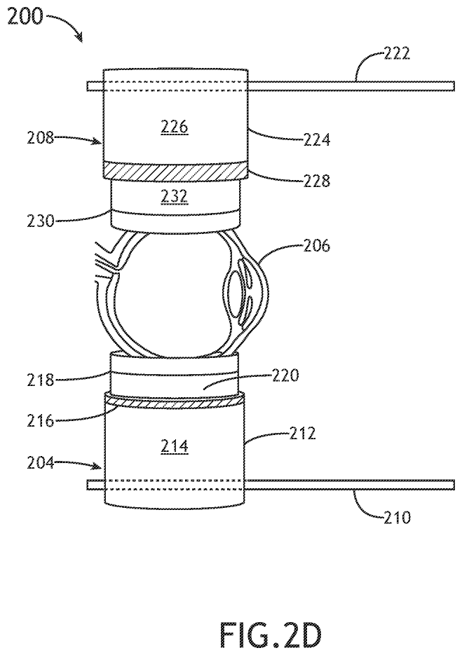

Based on the principles of the HIC 100, it was contemplated that a HIC-based iontophoresis system can safely apply higher current intensities than a conventional iontophoresis device to significantly enhance iontophoretic drug delivery efficiency without causing ocular tissue damage. FIG. 2A generally illustrates an ocular iontophoresis system 200 including a power source 202 coupled to an electrode-driven iontophoresis device 204, which may be loaded with a therapeutic solution to iontophoretically transport molecules (e.g., drug molecules) from the therapeutic solution across a surface to a target region. For example, the iontophoresis device 204 may be loaded with a drug solution to deliver drug molecules across an ocular surface to a target region in an eyeball 206. The iontophoresis system 200 may further include a counter electrode/device 208 to complete the circuit.

As shown in FIG. 2B, in HIC-based embodiments, the iontophoresis device 204 includes a chamber 212 containing a salt solution 214 (e.g., phosphate, sulfate, citrate, carbonate, or polyacrylate salt solution) and a chamber 218 containing a therapeutic solution 220, wherein chamber 212 and chamber 218 are separated by a hydrogel membrane 216 (e.g., PEG hydrogel matrix). When a carbon/metal electrode 210 is used to apply electrical current from the power source, the electrical current induces an ion current in the salt solution. This ion current is transmitted to (or induces a second ion current within) chamber 218 via the hydrogel membrane 216 while salt ions are stably contained in chamber 212 due to ATPS. By comparison, FIG. 2C illustrates a conventional iontophoresis device that applies electrical current from the carbon/metal electrode 210 directly to the therapeutic solution 220 in chamber 218, which may result in undesirable thermal and/or pH changes.

FIG. 2D illustrates a HIC-based embodiment of the system 200, wherein chamber 218 of the iontophoresis device 204 is configured to interface with an ocular surface (e.g., sclera, corneal epithelium, etc.) of an eyeball 206. When the electrode 210 applies an electrical current to chamber 212 of the iontophoresis device 204, an ion current is induced within in the salt solution 214. This ion current acts on (e.g., is transmitted to/through) chamber 218 to iontophoretically transport molecules (e.g., drug molecules) from the therapeutic solution 218 across the ocular surface to a target region (e.g., the vitreous or an intraocular region). In some embodiments, the system 200 includes a counter electrode device 208 that is similarly structured to mitigate thermal and/or pH changes at the interface between the eyeball 206 and the counter electrode. For example, the counter electrode device 208 may also include a chamber 224 containing a salt solution 226 (e.g., phosphate, sulfate, citrate, carbonate, or polyacrylate salt solution) that is at least partially bound by a hydrogel membrane 228 (e.g., PEG hydrogel matrix), wherein a counter electrode 222 (e.g., carbon/metal electrode) is coupled to chamber 224 to complete the circuit so that ion current flows from the iontophoresis device 204 through a portion of the eyeball 206 to the counter electrode device 208. In some embodiments, the counter electrode device 208 also includes a chamber 230 that is separated from chamber 224 by the hydrogel membrane 228. Chamber 230 may be configured to interface with a portion of the eyeball 206 and may contain a phosphate-buffered saline (PBS) solution 232 to further mitigate pH and/or thermal changes at the surface of the eyeball 206. In some embodiments, the system 200 can be used without therapeutic solution to apply electrical stimulus in the form of ion current to a portion of biological tissue (e.g., a portion of the eyeball 206). In such cases, chambers 218 and 230 may both contain PBS solution.

It is noted that, although the present disclosure focuses on transmission across ocular surfaces, the HIC-based iontophoresis systems/devices described herein may be used to deliver therapeutic solutions across a variety of biological surfaces to underlying target regions. Accordingly, the specific embodiments provided herein should be considered as non-limiting examples unless otherwise claimed.

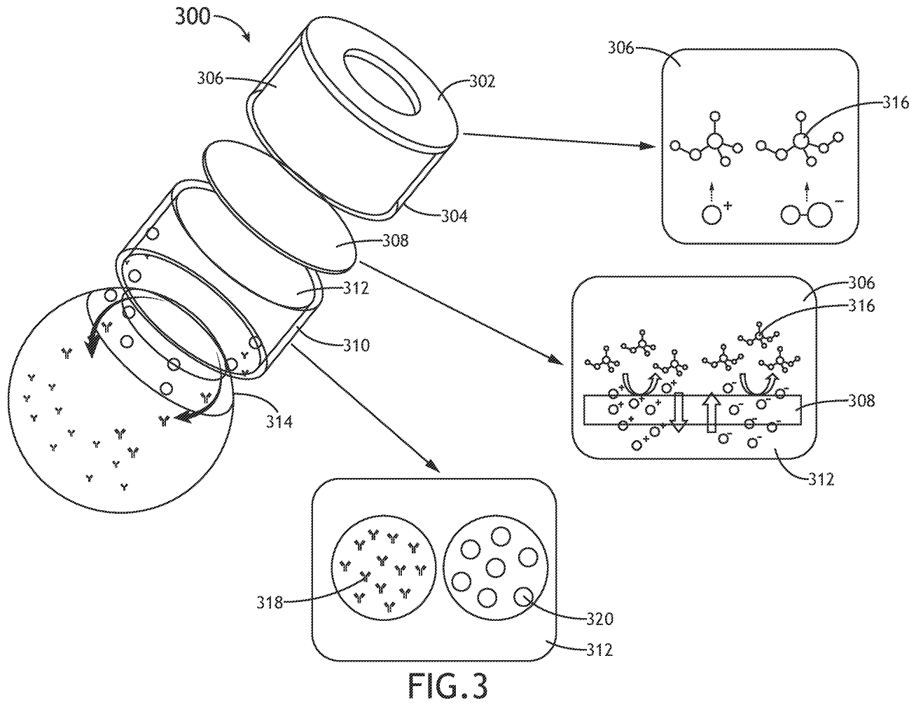

Referring now to FIGS. 3 through 10D, this disclosure presents an ion current-conducting ocular iontophoresis device 300 based on the HIC concept for efficacious intraocular delivery of macromolecular and nanoparticle drugs. It has been demonstrated that the HIC-based iontophoresis device 300 exhibits long-term stability in aqueous tissue-relevant environment. The HIC-based iontophoresis device 300 was capable of applying high-intensity DC currents to eyes with minimal physicochemical changes (temperature and pH) and ocular tissue damage. The ability to safely apply high-intensity currents allowed the HIC-based iontophoresis device 300 to significantly enhance the intraocular delivery of dextran (40 kDa), bevacizumab (a commonly used anti-VEGF agent for posterior segment diseases) and a nanoparticle-based sustained release formula of dexamethasone, compared to conventional ocular iontophoresis devices. As a result, therapeutically effective concentrations of bevacizumab and dexamethasone in nanoparticle formula can be non-invasively delivered into the target ocular tissues within 30 minutes. This will improve the safety of intraocular drug delivery and patient compliance. Furthermore, the HIC-based iontophoresis device 300 can be easily operated by caregivers without specially training, which will particularly benefit rural/home-bound patients and patients in developing countries who have limited access to specialists. By reducing the number of visits to major eye care centers by patients, the HIC-based iontophoresis device 300 may also reduce potential disease exposure, especially during a time of global pandemic, and overall healthcare cost.

Example Embodiment of the HIC-Based Iontophoresis Device

FIG. 3 is a schematic illustration of the HIC-based iontophoresis device 300, in accordance with an example embodiment of the present disclosure. In embodiments, the HIC-based iontophoresis device 300 includes or is coupled to an electrode 302 (e.g., a carbon/metal electrode) configured to apply electrical current to a chamber 304 containing a salt solution 306 (e.g., phosphate, sulfate, citrate, carbonate, or polyacrylate salt solution). In some embodiments, the electrode 302 is a circular or annularly shaped carbon electrode 302 coupled to chamber 304. The chamber 304 may include or may be coupled to a hydrogel membrane 308 (e.g., PEG hydrogel matrix) that defines a barrier for the salt solution 306 contained in chamber 304. The HIC-based iontophoresis device 300 further includes a chamber 310 containing a therapeutic solution 312 (e.g., a drug solution). In embodiments, chambers 304 and 310 are coupled together with the hydrogel membrane 308 being disposed between the chambers and configured to separate the salt solution 306 in chamber 304 from the therapeutic solution 312 in chamber 310. As shown in FIG. 3, the hydrogel membrane 308 may be ionically conductive and configured to permit certain ions (e.g., Na⁺, Cl⁻) to flow between the chambers 304 and 310 while salt ions 316 (e.g., phosphate, sulfate, citrate, carbonate, or polyacrylate salt ions) of the salt solution 306 are stably contained in chamber 304 due to ATPS.

Chamber 310 may be configured to interface with a portion of an ocular surface 314 (e.g., sclera, corneal epithelium, etc.) overlaying a target region (e.g., vitreous, posterior segment, or any other intraocular region). For example, the chamber 310 may have an opening or permeable/semi-permeable membrane configured to be placed into contact with the ocular surface 314. In some embodiments, chambers 304 and 310 may be cylindrical to provide a cylindrically stacked ocular iontophoresis device that can interface with a front portion of an eyeball much like a contact lens; however, other geometries may also be appropriate depending on the application.

When the HIC-based iontophoresis device 300 is in use, the electrode 302 is configured to apply electrical current from a power source to chamber 304, wherein the electrical current induces an ion current in the salt solution 306. This ion current is transmitted to (or induces a second ion current within) chamber 310 by via the hydrogel membrane 308 while salt ions are stably contained in chamber 304 due to ATPS. The ion current acts on (e.g., is transmitted to/through) chamber 310 to iontophoretically transport molecules (e.g., drug molecules, such as Anti-VEGF molecules 318, PLGA nanoparticles 320, etc., or any combination thereof) from the therapeutic solution 310 across the ocular surface 314.

A method of delivering a therapeutic agent (e.g., drug molecules) across the ocular surface 314 with the HIC-based iontophoresis device 300 may include, but is not limited to, the following steps: (1) disposing the salt solution 306 within chamber 304; (2) disposing the therapeutic solution 312 containing the therapeutic agent within chamber 310, wherein chamber 304 and chamber 310 are separated by the hydrogel membrane 308; (3) interfacing chamber 310 with the ocular surface 314; and (4) applying an electrical current to chamber 304 to induce an ion current in the salt solution 306, wherein the ion current acts on chamber 310 to iontophoretically transport the therapeutic agent from the therapeutic solution 312 across the ocular surface 314.

More generally, a method of delivering a current across an ocular surface 314 with the HIC-based iontophoresis device 300 (or a HIC electrode lacking chamber 310) may include, but is not limited to, the following steps: (1) disposing the salt solution 306 within chamber 304, wherein the chamber 304 is at least partially bound by the hydrogel membrane 308 that defines a barrier between the salt solution 306 and the ocular surface 314; and (2) applying an electrical current to the chamber 304 to induce an ion current in the salt solution 306, wherein the hydrogel membrane 308 is ionically conductive and configured to transmit the ion current across the ocular surface 314.

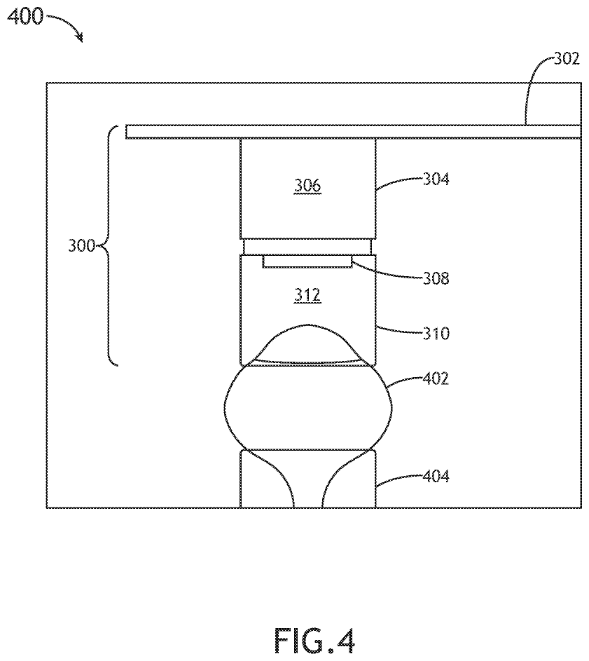

FIG. 4 shows an example embodiment of a HIC-based iontophoresis system 400 for performing ex vivo testing on an eyeball 402 (or portion thereof) with the HIC-based iontophoresis device 300. In the example illustrated in FIG. 4, the therapeutic solution chamber 310 of the HIC-based iontophoresis device 300 is interfaced with an ocular surface at the front of the eyeball 402 while a counter electrode device 404 is interfaced with the back of the eyeball 402. The counter electrode device 404 may be structured similarly to the HIC-based iontophoresis device 300 (either without a therapeutic solution chamber, or with a therapeutic solution chamber filled with PBS solution or another biocompatible buffer solution). The counter electrode device 404 serves to complete the circuit so that ion current flows from the HIC-based iontophoresis device 300 through a portion of the eyeball 402 to the counter electrode device 404. This facilitates iontophoretic transport across the ocular surface, whereby the ion current drives molecules (e.g., drug molecules or other therapeutic agents) from the therapeutic solution 312 across the ocular surface to an underlying target region in the eyeball 402.

In an in vivo/clinical implementation of the HIC-based iontophoresis device 300, the therapeutic solution chamber 310 may be placed into contact with a front/side portion of an eyeball and a counter electrode device may be placed into contact with a different front/side portion of the eyeball and/or into contact with surrounding tissue (e.g., a nearby portion of skin) to define a path for the ion current. For example, some ocular iontophoresis systems utilize a counter electrode applied to a patient's forehead.

During iontophoresis, the electron current from a DC current source is converted to ion current at the current source/HIC interface through EC reactions. In accordance with certain embodiments, the high-concentration salt solutions (e.g., phosphate, sulfate, citrate, carbonate, or polyacrylate salt solutions) in the HIC-based iontophoresis device 300 have high efficiency in buffering the pH changes, compared to physiological phosphate-buffered saline (PBS). They can also absorb the heat generated by the EC reactions. As a result, the pH and temperature changes do not affect the ocular tissues. The high-concentration salt solutions then route the ion current to the eye with higher conductivities compared to physiological saline, effectively reducing Joule heating. Furthermore, a unique aqueous two-phase separation (ATPS) between the PEG hydrogel and the salt solution is formed when their concentrations exceed specific thresholds. As a result of this phase separation, high-concentration salt ions (e.g., phosphate, sulfate, citrate, carbonate, or polyacrylate salt ions) are stably contained in chamber 304 with minimal diffusion into the PEG hydrogel, so the salt ions do not affect the drug solution or the ocular tissues.

Minimization of the Impact of pH Changes on the Eye

The Faradaic charge transfer between electron current-conducting electrodes and ion current-conducting media inevitably decomposes water molecules and generates pH changes when polarizable electrodes (e.g., platinum, carbon, etc.) are involved. Non-polarizable electrodes, such as silver/silver chloride, transfer charges through reactions between electrode material and soluble ions, so they do not decompose water molecules or generate pH changes. However, during high-intensity DC current application, the electrode material (e.g., AgCl) may be quickly depleted, after which water decomposition can happen. For this section, pH changes were estimated during high-intensity iontophoresis application and the amount of salt solution 306 (e.g., phosphate salt solution) to buffer these changes to maintain an ocular pH of 6.5 to 8.5, which is considered safe for ocular tissues. A worst-case scenario was used for estimation purposes, which assumed all electron transferred at the current source/HIC interface were used to decompose water molecule. Based on this assumption, the amount of salt (e.g., phosphate salt) required to maintain a safe ocular surface pH can be calculated using the Henderson-Hasselbalch equation, pH=pKₐ+log (Cₛ/Cₐₛ), where pKa is the negative logarithm of the acid dissociation constant for the salt (e.g., phosphate salt), and Cₛ and Cₐₛ are molarities of the weak acid and its conjugate base. Using an 100 mA, 15-minute long iontophoresis as an example, a total of 5.62×10²⁰ hydrogen ions and hydroxide ions will be generated at the anode and cathode interfaces, respectively. According to the Henderson-Hasselbalch equation, 0.9 mmol of disodium phosphate and sodium dihydrogen phosphate are required to react with these hydrogen/hydroxide ions to maintain an ocular surface pH between 6.5 and 8.5. This method was used to calculate the volume of anode and cathode salt solution in ex vivo drug delivery experiments.

Minimization of the Impact of Temperature Increase on the Eye

Two sources of heat are involved in the HIC-based iontophoresis device 300: (1) electrode overpotential-induced heat at the current source/HIC interface and (2) Joule heating. The electrode overpotential is the potential different between the actual voltage drop across the electrode/electrolyte interface and the thermodynamically required potential for EC reactions and charge transfer. For water electrolysis, the thermodynamically required potential is 1.48 V. Any additional potential above 1.48 V, i.e., overpotential, contributes to heat generation. The Joule heating is the resistive heating produced when current flows through a resistive media. For this section, heat generated during the iontophoresis procedure was estimated, as well as the volume of the liquid in the HIC-based iontophoresis device 300 that is required to maintain an eye temperature of less than 43° C., which is considered safe for ocular tissues.

Due to the complexity of the heat generation and transfer process, computer-aided finite-element analysis (FEA) was used to predict the heat generation and temperature distribution during the high-intensity iontophoresis procedure. The carbon electrode overpotential was measured using a voltage meter. The electrical conductivity of the salt solution 306 (e.g., phosphate salt solution) was measured using a LCR meter/conductivity meter. The electrical conductivity of ocular tissues, the heat transfer coefficients and the specific heat capacities were extracted from literature. The carbon electrode (3.8 cm2 contact area) overpotential at 100 mA was measured to be 4 V. In experiments, the highest temperature increase happened at the drug solution chamber 310, the ocular surface 314 that is in direct contact with drug solution 312, and the hydrogel membrane 308. The high-concentration salt solution 306 had lower temperature increase due to its high electrical conductivity. The inside of the eye also had lower temperature increase due to the lower current density inside of the eye when compared to the ocular surface 314 at the contact point with chamber 310.

Aqueous Two-Phase Separation (ATPS) Between PEG Hydrogel and High-Concentration Phosphate Salt Solution

As a critical feature in the HIC-based iontophoresis device 300, the function of phosphate-PEG ATPS is to contain the high-concentration salt solutions (e.g., salt solution 306) in the HIC-based iontophoresis device 300, so they can buffer pH changes, absorb heat, and conduct ion current with high conductivity, while having little impact on the surrounding ocular tissue environment. Therefore, the stability of the phosphate-PEG ATPS is of high importance to the correct functioning of the HIC-based iontophoresis device 300. Long-term stability of the phosphate-PEG ATPS and its long-term impact on surrounding tissue environment was examined. In embodiments, the PEG hydrogel (hydrogel membrane 308) was prepared by photo-crosslinking of a precursor solution containing 10% w/w PEG dimethacrylate (8 kDa) and 5% w/w PEG diacrylate (700 Da). This PEG formula exceeds the minimal PEG concentration required for the formation of phosphate-PEG ATPS and provides sufficient mechanical strength to support the weight of high-concentration salt solution in the HIC-based iontophoresis device 300. Saturated Na₂HPO₄ solution (0.6 M, pH=9.0±0.01) was used in the anode device to buffer the hydrogen ions generated by the anode reaction. Saturated Na₂HPO₄ solution was used here to achieve maximal hydrogen ion buffering capability and maximal electrical conductivity to minimize Joule heating. To buffer the hydroxide ions generated by cathode reaction, a NaH₂PO₄ solution is required. However, high-concentration NaH₂PO₄ solution typically has a pH that is much lower than 6.5 (e.g., 3 M NaH₂PO₄ solution has a pH of 3.9±0.01), which may cause ocular tissue damage. Therefore, a mixture solution containing 0.6 M NaH₂PO₄ and 0.48 M Na₂HPO₄ was used in the cathode device. This mixture had a pH of 6.42±0.01.

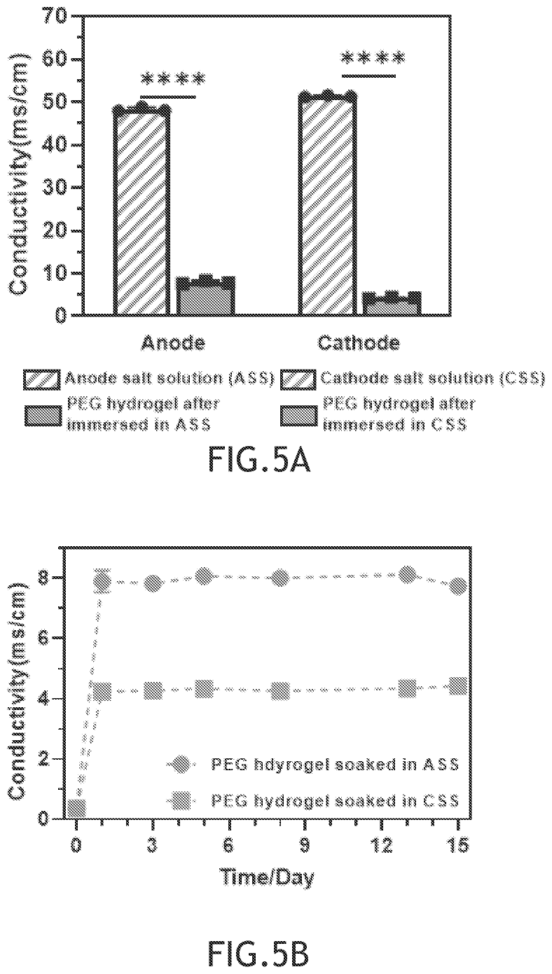

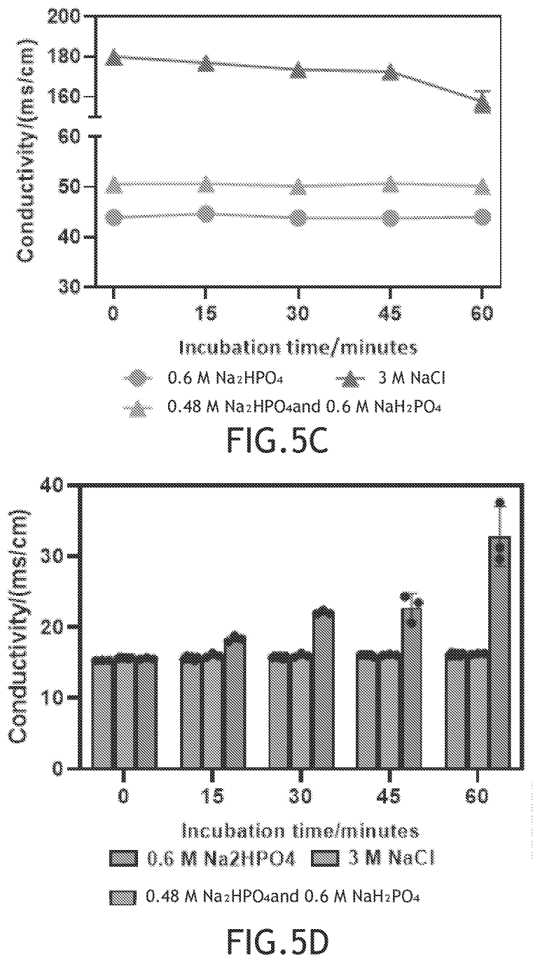

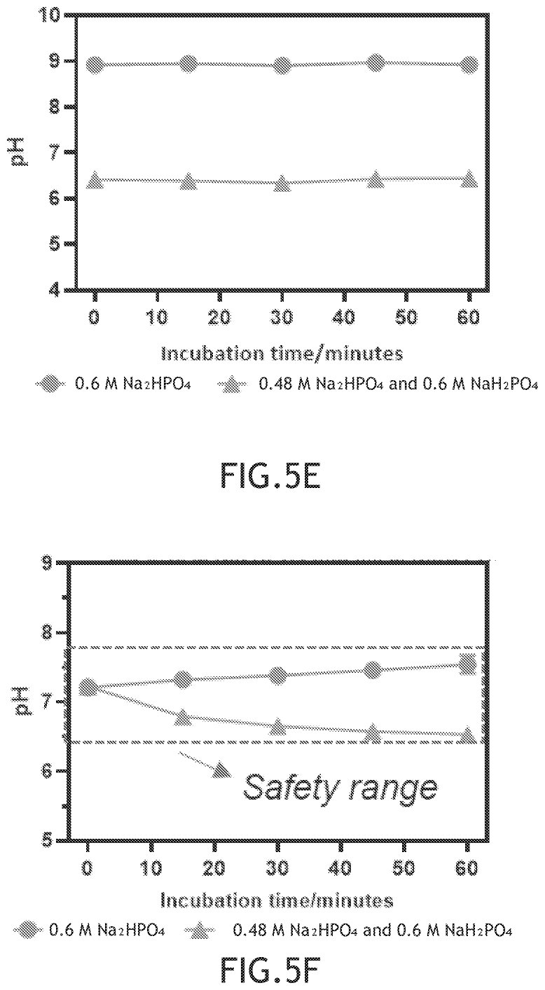

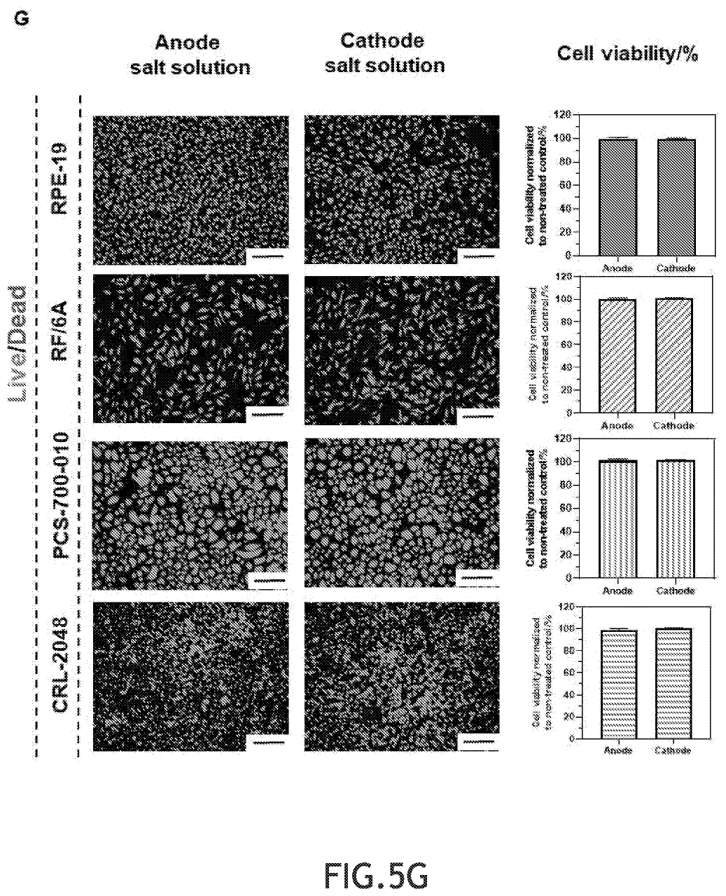

To evaluate ATPS stability, PEG hydrogel was immersed in anode and cathode phosphate salt solutions and measured its conductivity changes over a 2-week period. As can be seen in FIGS. 5A-5B, the PEG hydrogel maintained a conductivity that was 2-6 times lower than the conductivity of anode and cathode phosphate salt solution during the 2-week period. The fluctuation of the conductivity of PEG hydrogel was less than 20% during the 2-week period, indicating a stable phase separation between the PEG hydrogel and the phosphate salt.

Once the ATPS was formed, the high-concentration phosphate salt solution in the HIC-based iontophoresis device 300 had minimal impact on the surrounding environment. The HIC-based anode and cathode devices were then immersed in PBS and monitored the conductivity and pH changes of the phosphate salt solutions in the HIC-based iontophoresis device 300 and the PBS over a one-hour period. As a comparison, HIC anode and cathode devices filled with 3 M NaCl were also immersed in PBS. As shown in FIGS. 5C-5D, for HIC-based iontophoresis devices 300 filled with high-concentration phosphate salt solutions, neither the conductivity of the PBS nor the conductivity of the high-concentration phosphate salt solutions had minimal changes of less than 10%. However, when HIC-based iontophoresis devices 300 filled with 3 M NaCl was immersed in PBS, the conductivity of PBS increased by over 90%, while the conductivity of NaCl solution in the HIC-based iontophoresis device 300 decreased by over 10%. The pH of high-concentration phosphate salt solutions in the HIC-based iontophoresis device 300 and the pH of the PBS did not change during the one-hour immersion, as shown in FIGS. 5E-5F. The pH of PBS remained in the range of 6.5 to 8.5 that is considered safe for ocular tissues.

It was demonstrated that ATPS helped minimize the cytotoxic effects of the high-concentration phosphate salt solution in the HIC-based iontophoresis device 300. PEG hydrogel of HIC-based iontophoresis device 300 was immersed in in vitro cultures of corneal epithelium and endothelium cells, retinal pigmented epithelium cells, and choroid/retina endothelium cells for 1 h and measured the cell viability using LIVE/DEAD stain. As shown in FIG. 5G, the viability of all four cell types was minimally affected compared to control groups, showing the good cytocompatibility of the HIC-based iontophoresis device 300 enabled by ATPS.

Safety of High-Intensity Ion Current Application Using the HIC-Based Iontophoresis Device

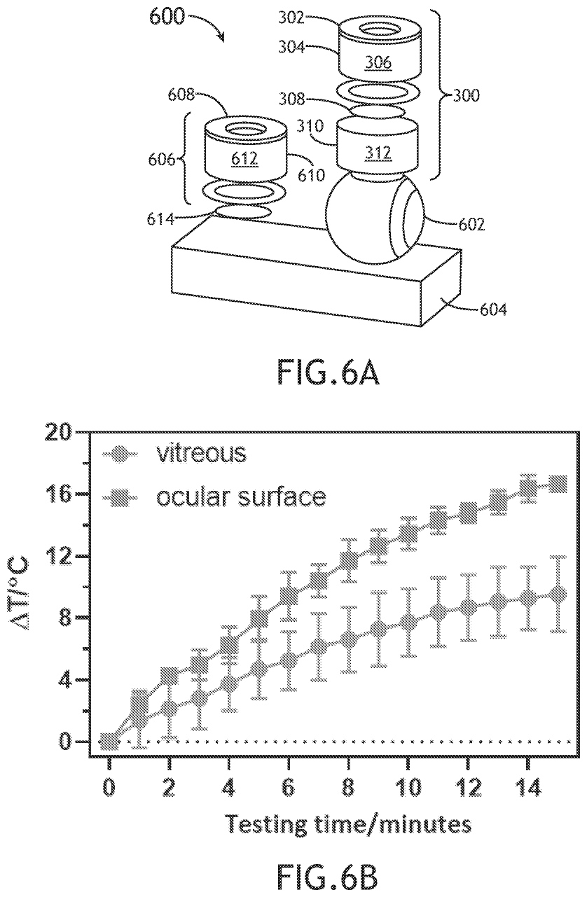

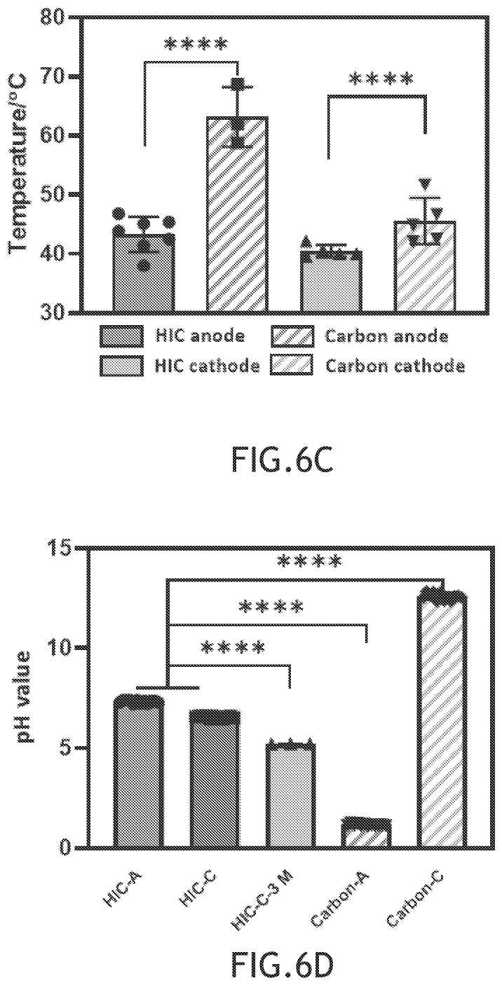

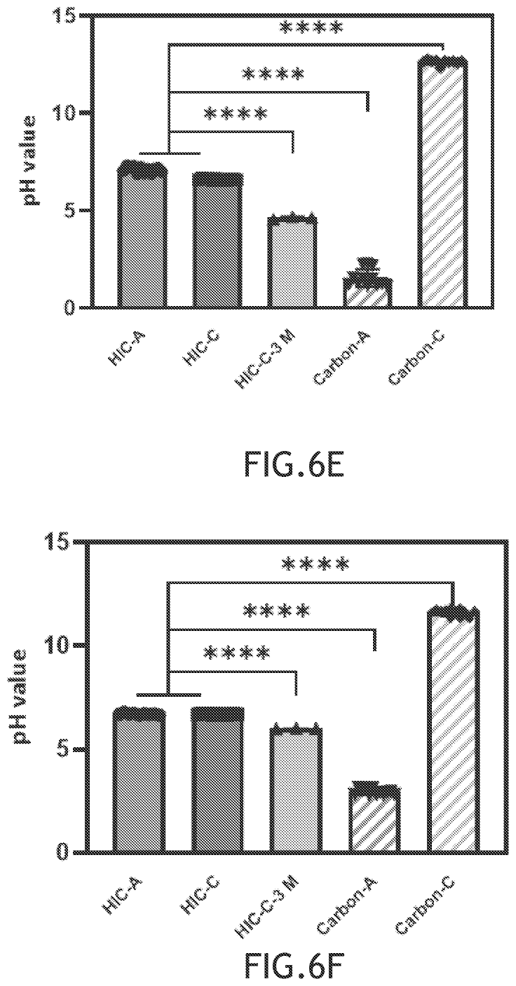

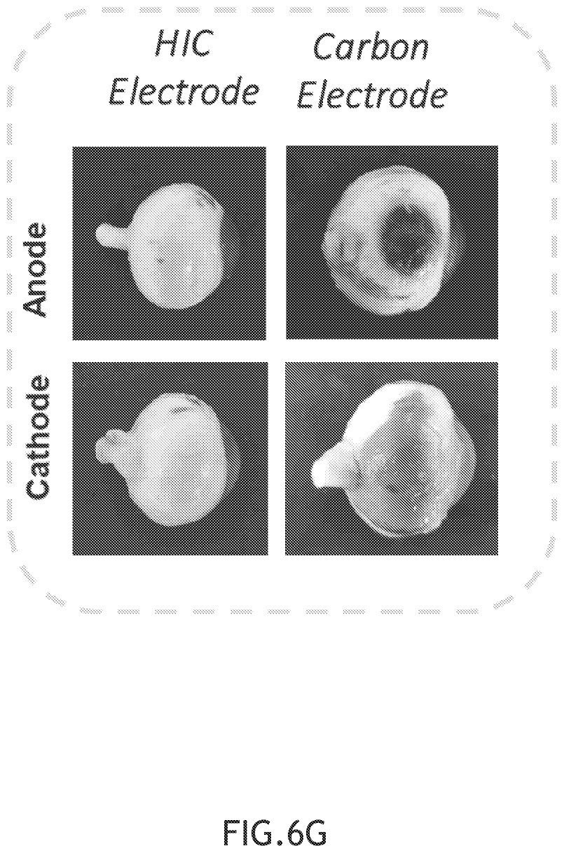

FIG. 6A shows an example embodiment of a HIC-based iontophoresis system 600 for performing ex vivo testing on an eyeball 602 (or portion thereof) with the HIC-based iontophoresis device 300. In the example illustrated in FIG. 6A, the therapeutic solution chamber 310 of the HIC-based iontophoresis device 300 is interfaced with an ocular surface at a side portion of the eyeball 602 while a counter electrode device 606 is interfaced with an opposite side of the eyeball 602 via an ion bridge 604 (e.g., a chamber filled with PBS or the like). The counter electrode device 606 may be structured similarly to the HIC-based iontophoresis device 300 (either without a therapeutic solution chamber, or with a therapeutic solution chamber filled with PBS solution or another biocompatible buffer solution). For example, in the embodiment illustrated in FIG. 6A, the counter electrode device 606 includes a chamber 610 coupled to a counter electrode 608. Chamber 610 contains a salt solution 612 (e.g., phosphate, sulfate, citrate, carbonate, or polyacrylate salt solution) and is at least partially bounded by a hydrogel membrane 614 that separates the salt solution 612 from interfacing components (e.g., ion bridge 604, or an intermediate chamber between chamber 610 and the ion bridge 604). The counter electrode device 606 serves to complete the circuit so that ion current flows from the HIC-based iontophoresis device 300 through a portion of the eyeball 602 to the counter electrode device 606 via the ion bridge 604. This facilitates iontophoretic transport across an ocular surface of the eyeball 602.

The safety of the HIC-based iontophoresis device 300 when applying high-intensity ion currents has been demonstrated using the system 600 illustrated in FIG. 6A. In testing, the HIC-based iontophoresis device 300 with PBS loaded in the drug solution chamber (chamber 310) was mounted on the equator of an isolated porcine eyeball (eyeball 602) and 100 mA DC current was applied. A conventional ocular iontophoresis device constructed by directly inserting a carbon electrode in a drug solution chamber was tested as a comparison. The current was applied continuously for 15 min and the temperature of the sclera surface was recorded using a thermocouple.

As shown in FIGS. 6B-6C, the temperature of the sclera surface was consistently increased due to the over-potential of electrolyte reaction and Joule's heat when conducting high current. However, the highest temperature of the sclera surface remained below 43° C., which is considered safe for ocular tissues. In contrast, the conventional iontophoresis device generated higher temperature increases, which reached 63.17±5.05° C. and 45.58±3.90 in the anode side and cathode side, respectively.

The pH was measured at the drug solution chamber (chamber 310), the sclera surface, and the vitreous fluid after the current application. As shown in FIGS. 6D-6F, when anode iontophoresis was applied to the equator of porcine eyeball using HIC-based iontophoresis device 300, the pH in the drug solution, sclera surface, and the vitreous fluid were 7.33±0.09, 7.13±0.17, and 6.72±0.04, respectively. When cathode iontophoresis applied to the equator of porcine eyeball using HIC-based iontophoresis device 300, the pH of these components were 6.55±0.06, 6.61±0.03, and 6.71±0.03, respectively. They were all remained in the safety range of pH that the ocular tissues can stand. However, the conventional iontophoresis device reduced the pH to lower than 3 at anode and increase the pH to higher than 11.5 at cathode. FIG. 6G shows images of porcine eyeball after high-intensity ion current application. Conventional iontophoresis device induced severe tissue damage due to chemical and thermal burns, which did not happen with the HIC-based iontophoresis device 300.

Enhancement of Drug Permeation Rate by High-Intensity Iontophoresis

There are two main mechanisms of iontophoresis: electrophoresis and electroosmosis. Both electrophoretically induced drug flux and electroosmotically induced drug flux are proportional to the applied current intensity:

$${J}_{eop} = ε ( - {z}_{j} {u}_{j} {C}_{j} \frac{d ψ}{dx} ) = ε ( - {z}_{j} {u}_{j} {C}_{j} \frac{Δ ψ}{h} ) = ε ( - {z}_{j} {u}_{j} {C}_{j} \frac{IR}{h} )$$

$${J}_{eos} = ε ( \pm {v}_{eff} C ) = ε ( \pm \frac{σ Δ ψ}{k η h} C ) = ε ( \pm \frac{σ IR}{k η h} C ) ,$$

where ε is the combined porosity and tortuosity factor of the membrane. ψ is the electrical potential, uⱼ is the effective elecromobility. v_{eff} is the average effective velocity due to convection results from electroosmosis. zⱼ is the charge number of the ion. C and x are the concentration and position of the permeant in the membrane, respectively. Δψ is the electrical potential applied across membrane. σ is the pore surface charge density. η is the velocity of the bulk solution. 1/k is the thickness of the electrical double layer. h is the membrane thickness. I is the current density and R is the electrical resistance normalized by the surface area of the membrane.

Therefore, it was contemplated that the high-intensity current enabled by the HIC-based iontophoresis device 300 can significantly enhance the iontophoretic drug delivery efficiency with a linear relationship. This hypothesis was tested by performing transscleral (porcine) drug delivery studies using a Franz cell setup and dextran (fluorescein isothiocyanate (FITC)-labeled, molecular weight=40 kDa) as a model drug.

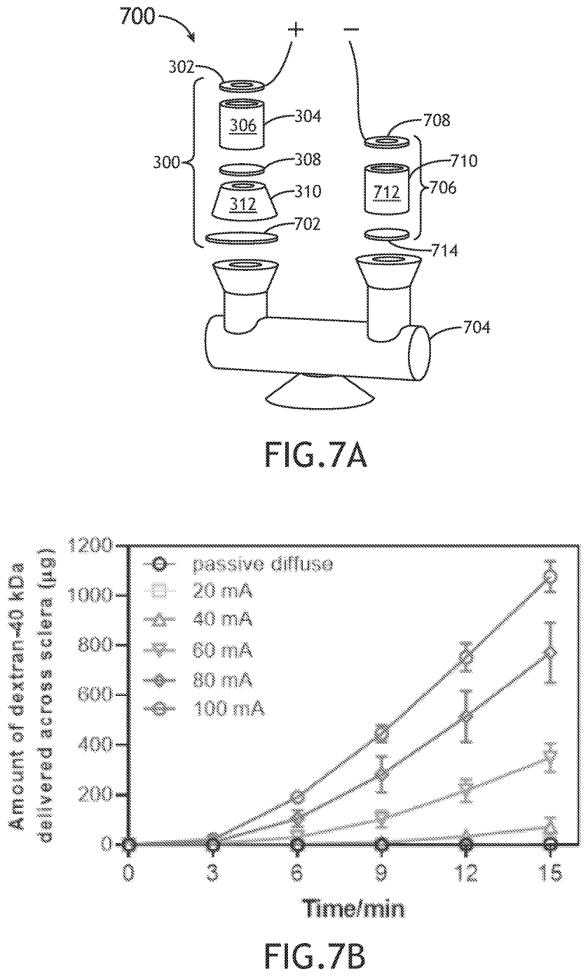

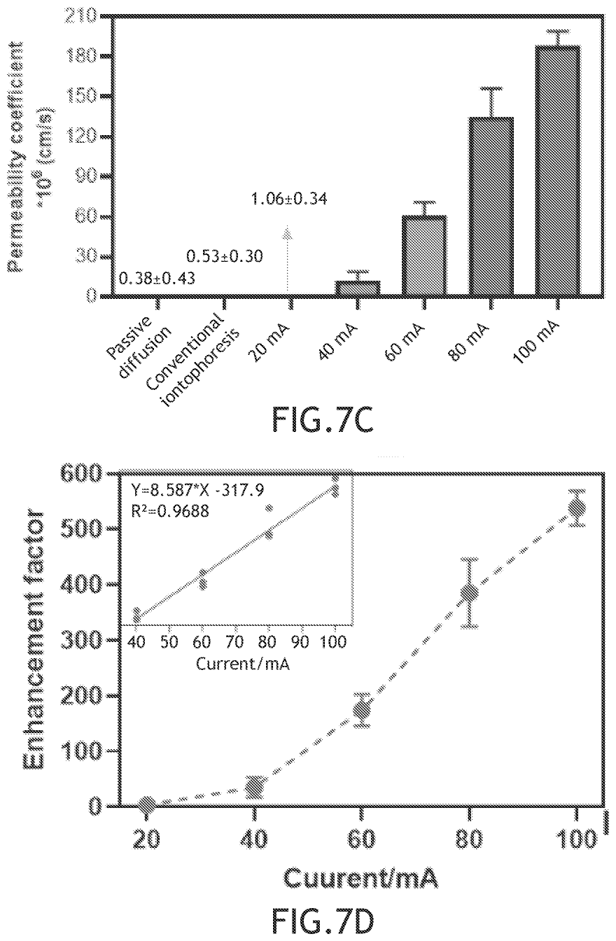

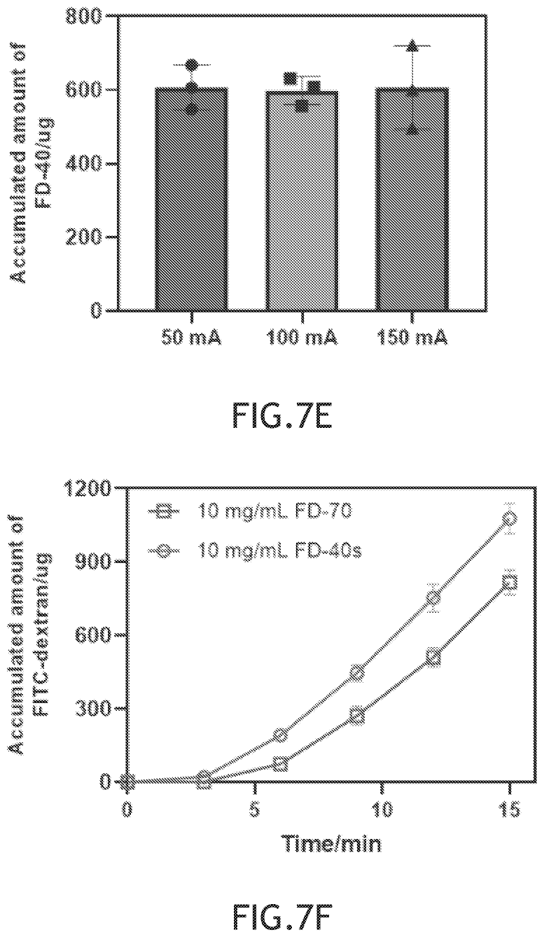

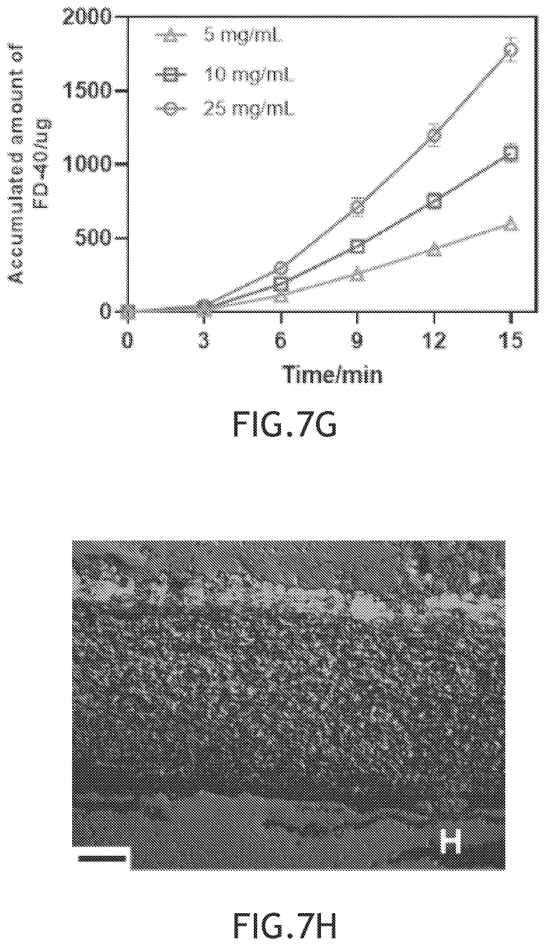

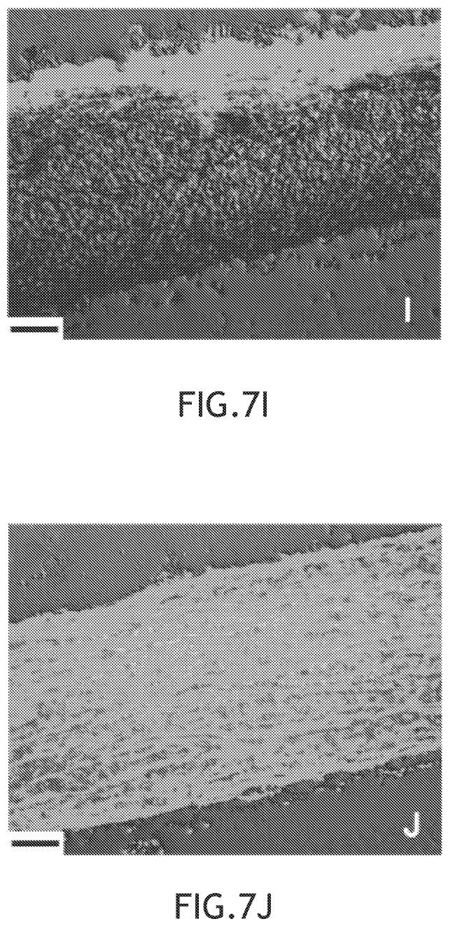

FIG. 7A shows an example embodiment of a Franz diffusion cell test system 700 for performing ex vivo testing on an eye surface tissue specimen 702 (e.g., sclera) with the HIC-based iontophoresis device 300. In the example illustrated in FIG. 7A, the therapeutic solution chamber 310 of the HIC-based iontophoresis device 300 is interfaced with one side of the eye surface tissue specimen 702 while a counter electrode device 706 is interfaced with an opposite side of the eye surface tissue specimen 702 via a receipt chamber 704. The counter electrode device 706 may be structured similarly to the HIC-based iontophoresis device 300 (either without a therapeutic solution chamber, or with a therapeutic solution chamber filled with PBS solution or another biocompatible buffer solution). For example, in the embodiment illustrated in FIG. 7A, the counter electrode device 706 includes a chamber 710 coupled to a counter electrode 708. Chamber 710 contains a salt solution 712 (e.g., phosphate, sulfate, citrate, carbonate, or polyacrylate salt solution) and is at least partially bounded by a hydrogel membrane 714 that separates the salt solution 712 from interfacing components (e.g., receipt chamber 704, or an intermediate chamber between chamber 710 and the receipt chamber 704). The counter electrode device 706 serves to complete the circuit so that ion current flows from the HIC-based iontophoresis device 300 through the eye surface tissue specimen 702 to the counter electrode device 706 via receipt chamber 704. This facilitates iontophoretic transport across the eye surface tissue specimen 702 into the receipt chamber 704.

Different current intensities were tested (e.g., 0 mA (i.e., passive diffusion), 40 mA, 60 mA, 80 mA, and 100 mA), and the amount of dextran-40 kDa was sampled in the receipt chamber 704 at different time points (3 min, 6 min, 9 min, 12 min, and 15 min) using a fluorescent plate reader. As shown in FIG. 7B, the dextran accumulation in the receipt chamber 704 had an initial lag phase in the first 3 minutes, where minimal dextran was detected in the receipt chamber 704 no matter the current intensity applied. Such a lag phase is well-documented in the literature and is attributed to the time required by drug molecules to penetrate sclera. After the lag phase, the dextran-40 kDa accumulation curves exhibited an approximately first-order relationship with the current intensity applied. It is worth noting that at 100 mA, the total amount of dextran-40 kDa transported across sclera at 15 minutes was 1,077.19±61.79 μg (FIG. 7B). This is close to or higher than the amount of anti-VEGF agent administered in each intravitreal injection (1.25 mg bevacizumab, or 2 mg aflibercept, or 0.5 mg ranibizumab is administered in each intravitreal injection). This demonstrates capability to deliver a therapeutic dose of macromolecule drug within a short period of iontophoresis application. This was further illustrated in FIG. 7C, where the permeation coefficient (Pc) of dextran-40 kDa at different current intensities at 15 minutes was shown. The Pc was significantly increased at higher current intensities and had a near linear relationship (R²=0.9688) with the current intensity applied for current intensities >40 mA. The drug permeation enhancement factors of the high current intensities applied over passive diffusion are shown in FIG. 7D. An over 500-time increase of Pc was achieved with 100 mA iontophoresis compared to passive diffusion, suggesting the strong drug permeation enhancement effect of high-intensity iontophoresis enabled by the HIC-based iontophoresis device 300.

The total amount of drug that permeate through tissue is directly proportional to the total amount of electrical charge applied. This was demonstrated by varying the current intensity and iontophoretic duration, while keeping the total charge applied the same. As can be seen in FIG. 7E, the three iontophoresis protocols tested (50 mA-30 min, 100 mA-15 min and 150 mA-10 min) all had 1,500 C in total charge applied and resulted in similar amount of dextran-40 kDa transferred to the receipt chamber. This result has important application. It allows precise control on the amount of drug delivered by changing the total charge supplied. It also allows for reduced iontophoresis duration by increasing current intensity, while achieving the same delivery efficiency. A shorter drug delivery duration enhances patient compliance to the treatment.

The dependence of iontophoretic drug delivery efficiency on the molecular weight of drug has also been studied. The transscleral delivery of fluorescently labeled dextran with molecule weight of 70 k (FD-70) was tested and compared to the results of FD-40. The amount of FD-70 permeated across sclera was in general lower than that of FD-40 during the 15-min iontophoresis at the same current intensity (FIG. 7F). The ratio of the amount of FD-40 permeated to the amount of FD-70 permeated at 15 min was 1.16. This is close to the ratio of the diffusion coefficient of FD-40 to the diffusion coefficient of FD-70, which is 1.22. This result is in good agreement with the previous demonstration that the iontophoretic permeability coefficient is directly proportional to the diffusion coefficient.

Lastly, the dependence of macromolecular drug delivery efficiency on the drug concentration loaded in the HIC-based iontophoresis device 300 was analyzed. Three different FD-40 concentrations were tested, including 5 mg/mL, 10 mg/mL, and 25 mg/mL. As shown in FIG. 7G, the amount of FD-40 delivered to the receipt chamber 704 increased with increasing drug concentration. However, the enhancement of drug delivery efficiency was not proportional to the drug concentration ratio. A 100% drug concentration increase from 5 to 10 mg/mL led to an 80% increase of total FD-40 permeated. A 150% drug concentration increase from 10 to 25 mg/mL led to a 65% increase of total FD-40 permeated. These results suggested that the permeability coefficient was lower at higher drug concentration, which is consistent with literature. FIGS. 7H-7J show the fluorescent dextran distribution in sclera after passive diffusion for 15 min (FIG. 7H), low-intensity iontophoresis at 7.5 mA/cm2 for 15 min (FIG. 7I), and high-intensity iontophoresis at 157 mA/cm2 for 15 min (FIG. 7J). The FD-40 penetrated the entire thickness of the sclera when high-intensity iontophoresis was used, while for low-intensity iontophoresis and passive diffusion the FD-40 was only present near the surface of the sclera. These images show that high-intensity iontophoresis provided higher electrical driving force to enhance the penetration of macromolecular drugs.

Macromolecule Delivery to the Posterior Segment Using the HIC-Based Iontophoresis Device

In the Franz cell study described above, it was demonstrated that the significant enhancement effect of the HIC-enabled high-intensity iontophoresis on transscleral macromolecular drug delivery efficiency. A step further was taken to determine the efficacy of the HIC-based iontophoresis technology in enhancing the macromolecular drug delivery to posterior segment using excised rabbit whole eyes. Rabbit eyes were used because they have similar size as human eyes. For this ex vivo study, the anode (i.e., HIC-based iontophoretic device 300) was attached to the front of the eye. The cornea was covered by a plastic film, so the drug molecule could only enter the eye at pars plana. The cathode (i.e., counter electrode device) was attached to the back of the eye.

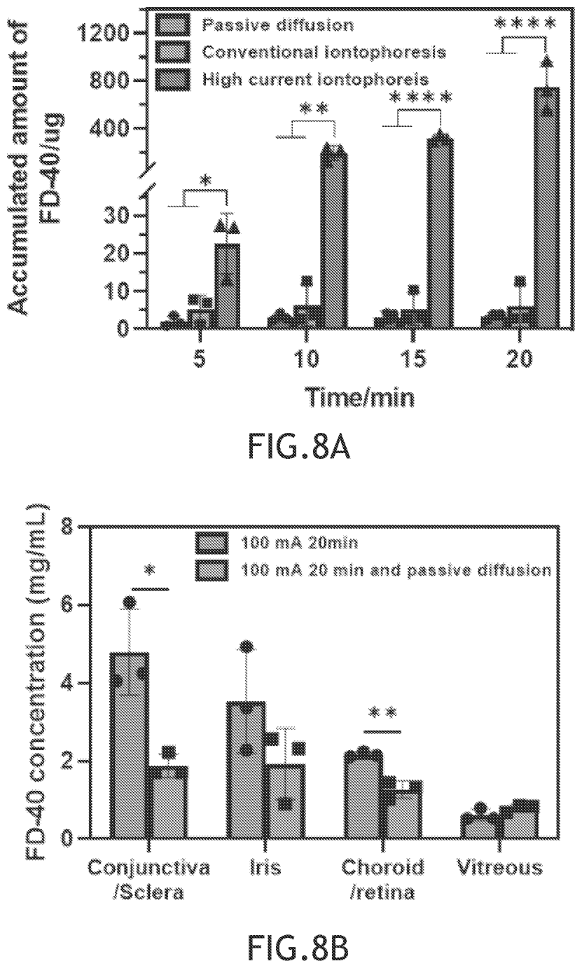

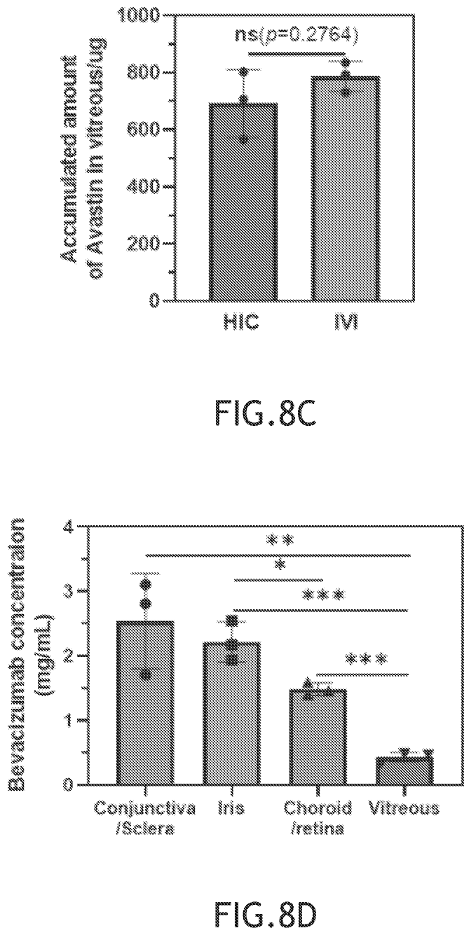

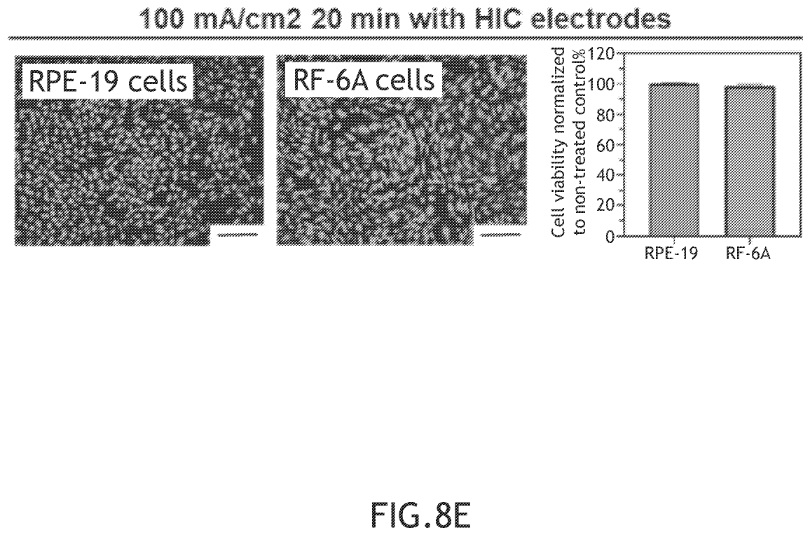

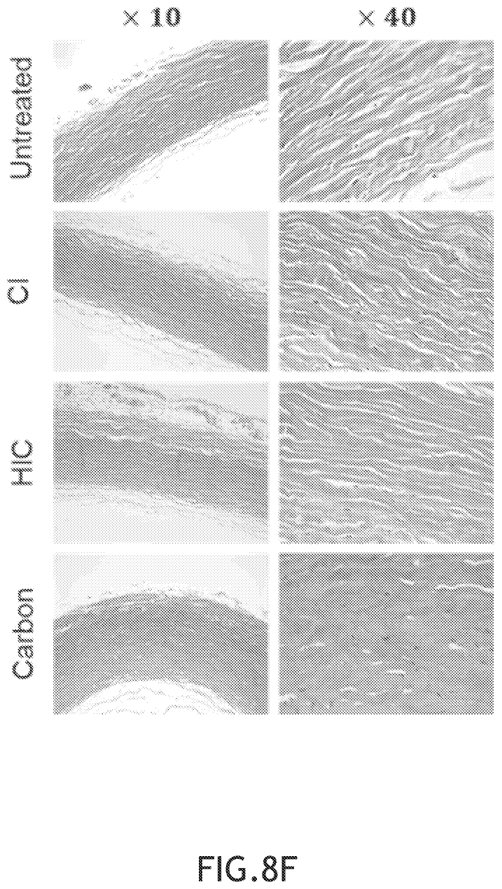

Iontophoretic delivery of FD-40 was first tested. 100 mA current was applied for 5 min, 10 min, 15 min, and 20 min. The total amount of FD-40 delivered into the eye and its distribution in different tissue layers were measured at different time points. FIG. 8A shows the total amount of FD-40 delivered into the eye, which as expected increased with increasing iontophoresis duration. Compared to passive diffusion and low-intensity iontophoresis at 7.5 mA/cm2, high-intensity iontophoresis enhanced the FD-40 delivery efficiency by 217 and 122 times. FIG. 8B shows the concentration of FD-40 in different ocular tissue compartments after 20 min iontophoresis at 157 mA/cm2. As can be seen, the FD-40 penetrated the sclera-choroid-retina tri-layer and entered the vitreous. The total amount of FD-40 delivered into the vitreous at 20 min was more than 600 μg, which is similar to the amount of bevacizumab (600 μg) and higher than the amount of ranibizumab (243 μg) administered into the vitreous by intravitreal injection. After iontophoresis, there was a high concentration of FD-40 in conjunctiva/sclera. This could potentially serve as a local drug “depot” to continuously transport drug to deeper tissue layer after iontophoresis was stopped. This phenomenon was demonstrated by allowing FD-40 to diffuse within the eye for 21 hours after the 20-min iontophoresis. The FD-40 concentration was measured in different tissue compartments at 21-hour post iontophoresis (FIG. 8B). A higher FD-40 concentration was detected in vitreous and choroid/retina. This drug “depot” effect could potentially prolong the effective time window of anti-VEGF agents.

In light of promising results from the FD-40 posterior segment delivery study, the inventors sought to determine whether the HIC-based iontophoresis device 300 could deliver a therapeutically effective concentrations of bevacizumab, the most commonly used ophthalmic anti-VEGF agent, into the posterior segment. 100 mA iontophoresis was applied with 25 mg/mL bevacizumab loaded in the HIC-based iontophoresis device 300 for 20 min. It was found that 692.02±119.24 μg bevacizumab was delivered into the vitreous (FIG. 8C), which was close to the amount delivered by intravitreal injection (786.72±52.46 ug). Similar to the FD-40 distribution in different tissue layers after 100 mA 20 min transscleral iontophoresis, the bevacizumab was penetrated into the vitreous from sclera-iris-choroid-retina route (FIG. 8D).

As previously mentioned, the primary concern of using high-intensity iontophoresis is its potential damage to ocular tissues. It was demonstrated above that the HIC-based iontophoresis device 300 induced minimal temperature and pH changes that were within the safe range for ocular tissues. Here, the impact of high-intensity ion current application on the integrity of ocular tissues was determined using freshly excised rabbit eyes and the viability of in vitro cultured ocular cells. The results demonstrated that after treated by high-intensity ion current (157 mA/cm2 for 20 minutes) applied by the HIC-based iontophoresis device 300, ocular tissue structure remained intact (FIG. 8F) and had no appreciable damage compared to the untreated control. However, ocular tissue treated with conventional iontophoresis device at the same current intensity and duration was severely damaged. Next, 157 mA/cm2 current was applied to in vitro cultured human retinal pigment epithelial cells (ARPE-19) and monkey choroid-retina endothelial cells (RF/6A) cells for 20 min using the HIC-based iontophoresis device 300. Cell viability was quantified using LIVE/DEAD stain (FIG. 8E). The results demonstrated that the viability of both cells was not affected by high-intensity ion current applied by the HIC-based iontophoresis device 300.

Intracorneal Delivery of Macromolecules Using the HIC-Based Iontophoresis Device

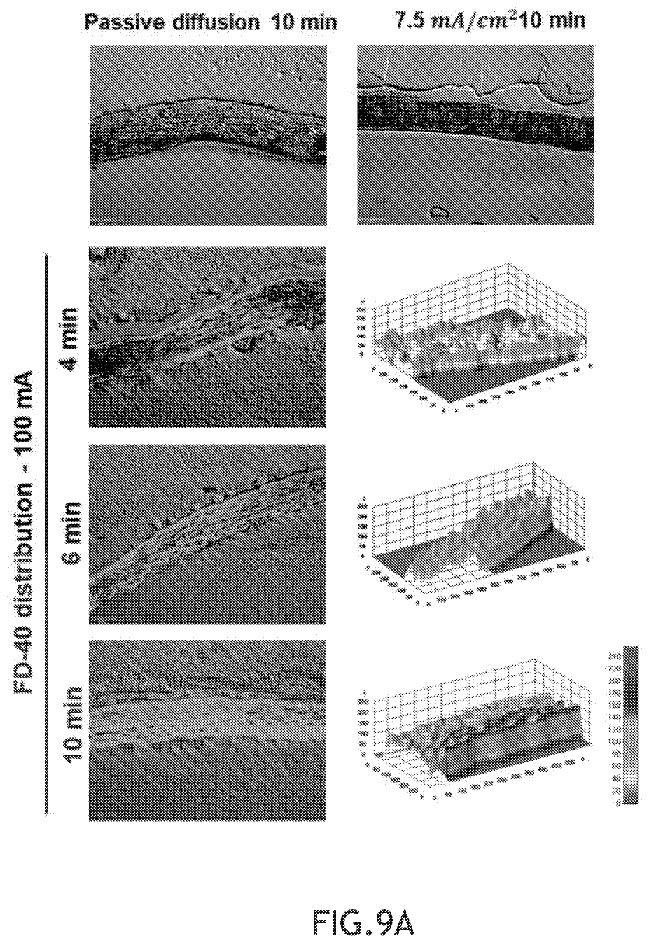

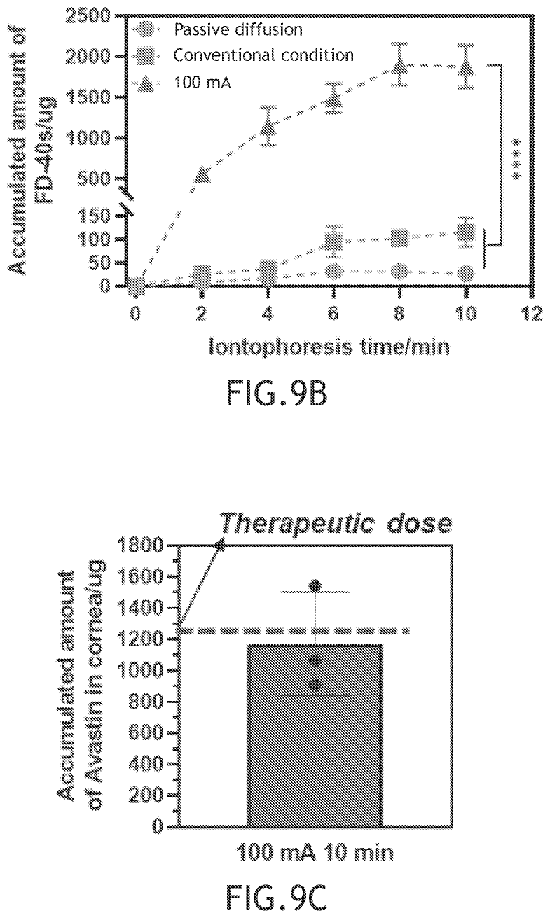

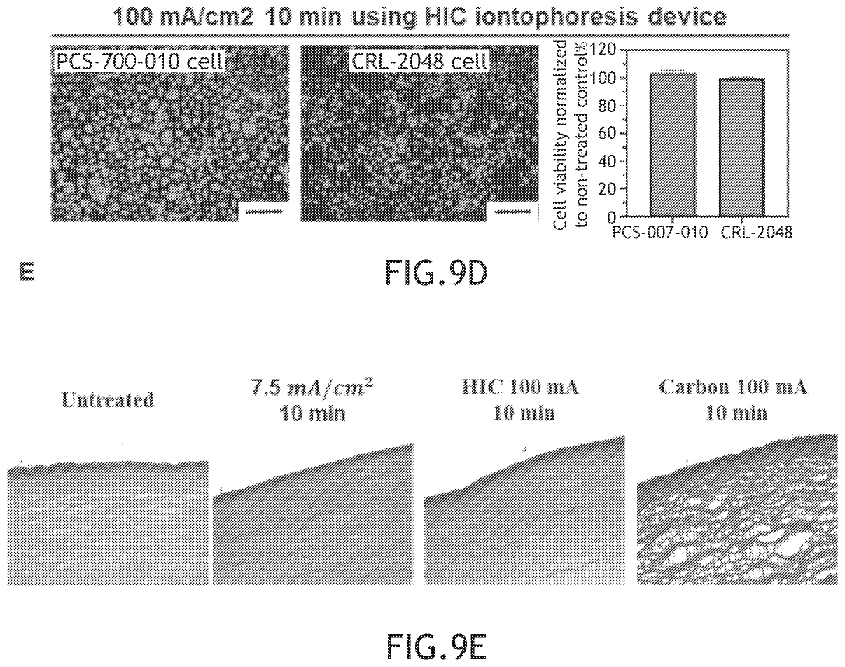

In light of promising ex vivo posterior segment macromolecule delivery results, the inventors sought to determine the utility of the HIC-based iontophoresis device 300 in delivering macromolecules to the anterior segment, particularly the cornea. Corneal neovascularization is one of the major ocular diseases occurred in the anterior eye segment, particularly in the stromal layer of cornea. It is commonly treated by subconjunctival or intrastromal injection of anti-VEGF, which can lead to various complications. The tight junctions of the corneal epithelium presents a major barrier to macromolecular drugs, so their diffusion to deeper corneal layers (the stromal and endothelial layers) is limited. It has been reported that the passive diffusion was not effective in delivering dextran to the mouse corneal epithelium, and the penetration improvement was also limited by using conventional iontophoresis devices. High-intensity iontophoresis of FD-40 (157 mA/cm2) was applied on cornea using the HIC-based iontophoresis device 300 for different durations from 2 to 10 minutes. The drug solution chamber (chamber 310) was filled with 25 mg/mL FD-40. High-intensity iontophoresis using the HIC-based iontophoresis device 300 was compared to passive diffusion and low-intensity iontophoresis at 7.5 mA/cm2 applied by the HIC-based iontophoresis device 300. At the end of the iontophoresis, the corneal tissues were examined under fluorescent microscope to visualize the distribution of FD-40. The total amount of FD-40 in cornea was also measured. As shown in FIG. 9A, FD-40 was not visible in the cornea after passive diffusion for 10 min due to the barrier function of the corneal epithelium. Low-intensity iontophoresis (7.5 mA/cm2) for 10 min delivered more FD-40 into the corneal, but they mainly accumulated near the surface of the cornea. When high-intensity iontophoresis at 100 mA (77 mA/cm2) was applied, FD-40 penetrated the entire thickness of cornea with visible FD-40 in the stromal and endothelial layers in as early as 4 min.

The accumulated amount of FD-40 in cornea as a function of iontophoresis duration was shown in FIG. 9B. High-intensity iontophoresis using the HIC-based iontophoresis device 300 significantly improved FD-40 delivery efficiency compared to passive diffusion and low-intensity iontophoresis. The accumulated amount of FD-40 after 10 min high-intensity iontophoresis was 1876.46±264.29 μg, while only 29.93±16.57 μg and 115.34±30.90 μg were delivered to cornea by passive diffusion and low-intensity iontophoresis, respectively. It is worth noting that the accumulated amount of FD-40 in the cornea after 4 min high-intensity iontophoresis reached 1142.90±233.86 μg, which is close to the intrastromal injection dose of bevacizumab (1.25 mg) for treating corneal neovascularization. This demonstrated the potential of the HIC-based iontophoresis device 300 to deliver a therapeutic dose of anti-VEGF agent to cornea within a short period of time. To demonstrate this, the intracorneal bevacizumab delivery efficiency of high-intensity iontophoresis at 100 mA (77 mA/cm2) was tested. 10 mg/mL bevacizumab was loaded in the drug solution chamber (chamber 310). The results demonstrated that 1170.22±331.51 μg bevacizumab was delivered into cornea under 100 mA ionic current after 10 min, which is comparable to the therapeutic dose of current anti-VEGF injection therapy.

Next, the impact of high-intensity ion current application by the HIC-based iontophoresis device 300 on corneal tissue integrity was examined using freshly excised rabbit eyes and the viability of in vitro cultured corneal cells. As shown in FIG. 9E, when the HIC-based iontophoresis device 300 was used to apply the high-intensity ion current, the stromal layer remained structurally intact and no appreciable difference from the untreated control could be discerned. In contrast, when conventional iontophoresis device was used to apply the same current intensity, the tissue structure of the stromal layer was severely disarrayed and damaged. Moreover, high-intensity ion current application by the HIC-based iontophoresis device 300 (157 mA/cm2 for 10 minutes) did not induce significant changes of the viability of in vitro cultured human corneal epithelial cells (PCS-700-010) and bovine corneal endothelial cells (CRL-2048) compared to the untreated control (FIG. 9D). These results indicated the safety of HIC-based iontophoresis device 300 operated with high current intensity for intracorneal drug delivery applications.

Intraocular Delivery of Nanoparticles Using the HIC-Based Iontophoresis Device

The nanoparticle ophthalmic drug formula has several advantages over free-form drugs, including the capability of sustained release, improved drug stability, and the capability of incorporating both hydrophilic and hydrophobic drugs. However, nanoparticles have low permeation rate in ocular tissues due to their large sizes. As a result, nanoparticle ophthalmic drugs are most commonly administered through injection, which can cause potentially blinding ocular tissue damage and adverse impact on patient compliance. The inventors sought to determine the efficacy of HIC-based iontophoresis device 300 operated with high current intensity to enhance the intraocular delivery of nanoparticle ophthalmic drugs. Dexamethasone-loaded poly(lactic-co-glycolic acid) (PLGA) nanoparticle was used as a model drug.

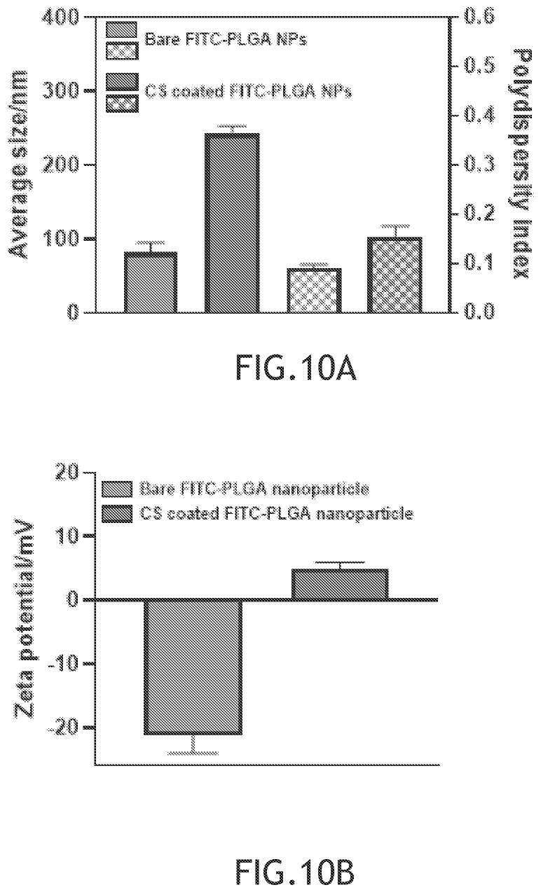

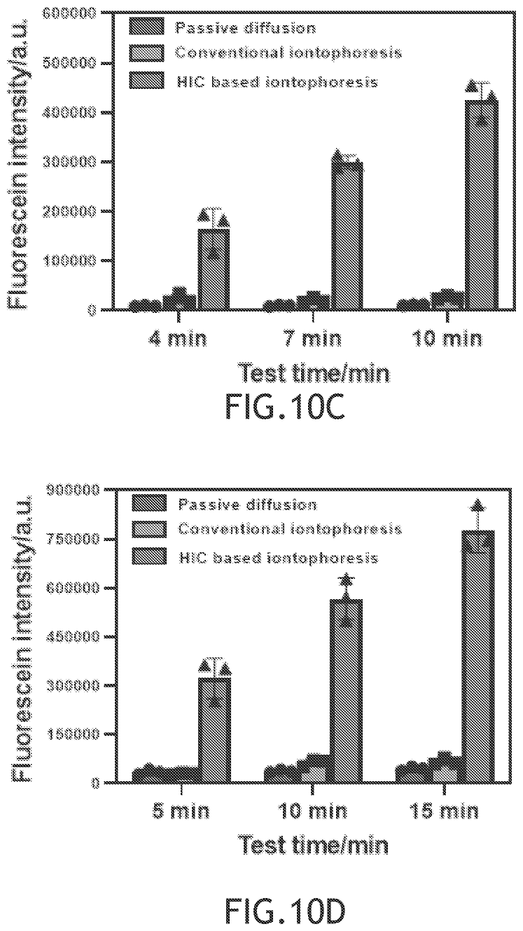

In this study, PLGA nanoparticles were fabricated and coated with positively charged chitosan. FTIC was loaded in the nanoparticles as a small molecular drug model. As shown in FIGS. 10A-10B, 243.23±9.85 nm chitosan coated FITC-PLGA nanoparticles were successfully fabricated using modified method, with polydispersity index of 0.16±0.02. The surface charge of PLGA nanoparticles was changed from −21.28±2.80 mV to 5.00±0.97 mV after chitosan coated on the surface. Positively charged nanoparticles were then used for iontophoresis test using high ionic current conducted by the HIC-based iontophoresis device 300. Both cornea and sclera were considered as the target ocular tissues. 100 mA DC current was conducted for different time points (i.e., 4 min, 7 min, and 10 min for corneal iontophoresis, 5 min, 10 min, and 15 min for scleral iontophoresis). By measuring the fluorescein intensity of nanoparticles extracted from cornea and sclera respectively, the delivery efficiency was analyzed and compared to those under passive diffusion and low current conducted iontophoresis. Both corneal iontophoresis and scleral iontophoresis results (FIGS. 10C-10D) confirmed that much higher delivery efficacy of PLGA nanoparticles were obtained by using high ionic current with the HIC-based iontophoresis device 300. Besides, the targeted tissues were triply washed with sodium chloride (0.9%) before nanoparticle extraction process to mimic the tear turnover function. Thus, it is contemplated that the positively charged PLGA nanoparticles were bonded with targeted tissues and would be helpful to sustained release of loaded drugs.

Example Embodiment of an Ocular Lens Embedded HIC-Based Iontophoresis Device

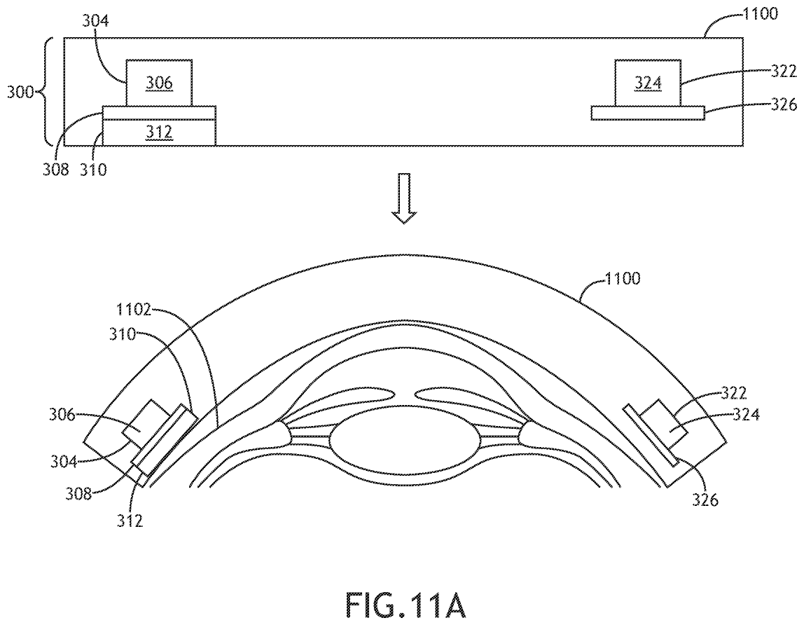

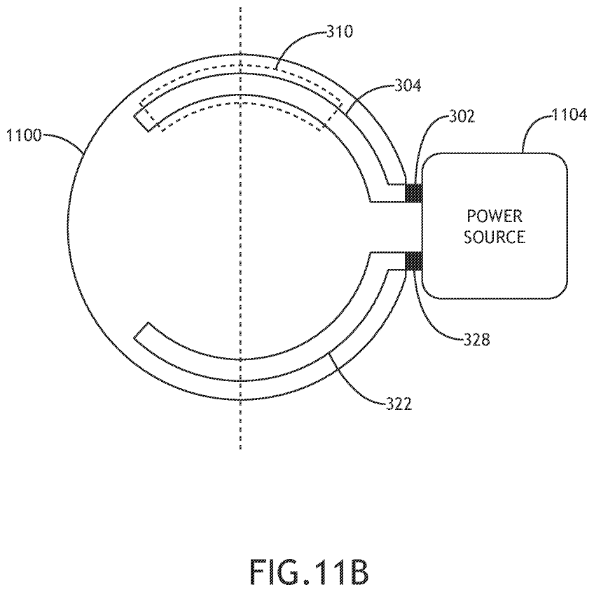

As shown in FIGS. 11A and 11B, the HIC-based iontophoresis device 300 may be embedded within an ocular lens 1100 (e.g., a contact lens) that can be applied to an eyeball 1102 to facilitate iontophoretic drug delivery across an ocular surface (e.g., sclera, corneal epithelium, etc.) at the front of the eyeball 1102. This lens contained iontophoresis system is a water-stable, hydrogel-based circuit capable of conducting ion currents. Within the lens embedded HIC-based iontophoresis device 300, high concentration salt solution-filled channels with hydrogel matrices are used to conduct ion currents. An aqueous two-phase system formed between the hydrogel and salt solutions (e.g., sodium chloride, sodium phosphate, potassium chloride, lithium chloride, etc.) stabilizes salt ions in the channels so their diffusion into the hydrogel or surrounding aqueous medium is minimized. This allows the hydrogel to permit ion currents to pass, so electrical stimulation can be delivered to the tissues.

In embodiments, chamber 304, hydrogel membrane 308, and chamber 310 are stacked within the ocular lens 1100, wherein chamber 304 defines a channel containing the salt solution 306 and chamber 310 defines another channel containing the therapeutic solution 312. As shown in FIG. 11B, the channels may be shaped as annular segments conforming to a contour of the eyeball 1102; however, other geometries may be appropriate as well (e.g., linear channels, zigzagged channels, etc.).

In some embodiments, the ocular lens 1100 is formed from a hydrogel. For example, the ocular lens 1100 may be formed from the same hydrogel as the hydrogel membrane 308. In this regard, the hydrogel membrane 308 may simply be a portion of the ocular lens 1100 disposed between chambers 304 and 310. Alternatively, the ocular lens 1100 may be formed from a different hydrogel or different biocompatible material. In embodiments, the channels/chambers may be defined by the ocular lens 1100 structure. For example, the ocular lens 1100 may include the channels/chambers etched or molded within the material (e.g., hydrogel) making up the ocular lens 1100. Alternatively, the channels/chambers can be separately manufactured and then embedded within the ocular lens 1100, or built into the ocular lens 1100 (e.g., by 3D printing or another material deposition technique).

The lens embedded HIC-based iontophoresis device 300 may further include a counter electrode channel embedded within the ocular lens 1100 to complete the circuit. The counter electrode channel may also include a chamber 322 containing a salt solution 324 (e.g., phosphate, sulfate, citrate, carbonate, or polyacrylate salt solution) that is at least partially bound by a hydrogel membrane 326 (e.g., PEG hydrogel matrix), wherein a counter electrode 328 (e.g., carbon/metal electrode) is coupled to chamber 322 to complete the circuit so that ion current flows from the electrode channel defined by chamber 304 through a portion of the eyeball 1102 to the counter electrode channel defined by chamber 322. In some embodiments, the counter electrode channel also includes another chamber that is separated from chamber 322 by the hydrogel membrane 326. The chamber adjacent to chamber 322 may be configured to interface with a portion of the eyeball 1102 and may contain a PBS solution or any other inert solution/buffer to further mitigate pH and/or thermal changes at the surface of the eyeball 1102.

The HIC channels defined by chambers 304 and 322 may be coupled to a power source 1104, respectively, by electrodes 302 and 328 (e.g., carbon/metal electrodes). In some embodiments, the HIC channels may be used to perfuse therapeutic agents (e.g., drug molecules) in either direction depending on whether the therapeutic solution 312 is loaded next to chamber 304 or chamber 322. In further embodiments, chamber 310 is a fluid-defined chamber that is defined by a gel loaded with drug solution 312, such that the drug loaded gel (chamber 310) can be disposed next to either HIC channel (i.e., next to chamber 304 or chamber 322). By engineering this biocompatible circuit design into a contact lens, drug (e.g., anti-VEGF, antibiotics, antifungals) loaded gel (chamber 310) can be positioned by either HIC channel (i.e., adjacent to chamber 304 or chamber 322) and perfused across the inducible ion gradient in order to perfuse drug into the posterior segment of the eyeball 1102. This device may facilitate high efficiency drug delivery despite static barriers (e.g., cornea, lens, and choroid) and dynamic barriers (e.g., blood and lymph flow).

Example Embodiment of a HIC-Based Electrical Stimulation Device for Wound Healing

At a cellular level, electrical stimulation can alter cellular activities, which further changes cellular functions, such as contraction, migration, and proliferation. The ability of electrical stimulation to provide a non-pharmacological way to facilitate cell migration has been studied with regard to its effects on wound healing. Many cell types migrate directionally in direct current (DC) electrical field, a phenomenon called electrotaxis. It has been demonstrated that electrotaxis increased the speed of closure of in vitro scratch wounds, suggesting strong therapeutic potentials of electrical stimulation. However, in vivo studies that tested the efficacy of electrical stimulation for wound healing showed inconsistent outcomes. For example, out of eleven randomized controlled clinical trials (RCTs) conducted from 1985 to 2010, five showed that pulsed or continuous DC electrical stimulation significantly increased the wound healing rate compared to controls (p<0.05) (14-18). One RCT showed significantly increased wound healing rate by pulsed DC electrical stimulation (p<0.05), but the number of wounds healed was fewer than control at the end of the study (p>0.05). Five other RCTs showed insignificant (p>0.05) or no improvement on wound healing by pulsed DC electrical stimulation compared to controls (20-24). A careful review of these studies revealed one common problem that is likely to be responsible for such inconsistent outcomes: the time when electrical stimulation was applied was too short due to the use of pulsed voltage. For most studies, the voltage was applied for less than 4 minutes per day. This was not sufficient for electrotaxis-induced scratch wound healing, which requires at least 2 hours of electrical stimulation.

In order to apply electrical stimulation safely for a sufficient amount of time and a high enough current intensity to improve the rate of wound healing, there is a need for improved circuit design that will not damage biological tissue or cause intolerable pain/burning sensation. To address this need, a HIC-based electrical stimulation device is disclosed.

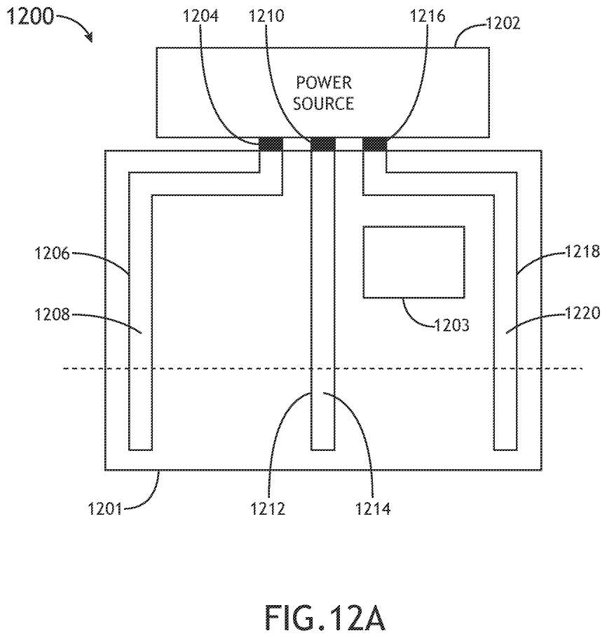

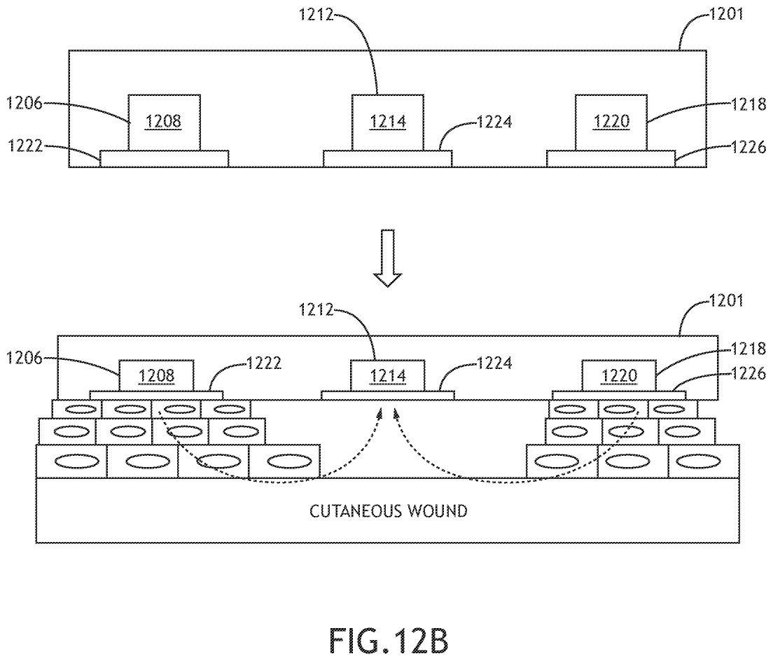

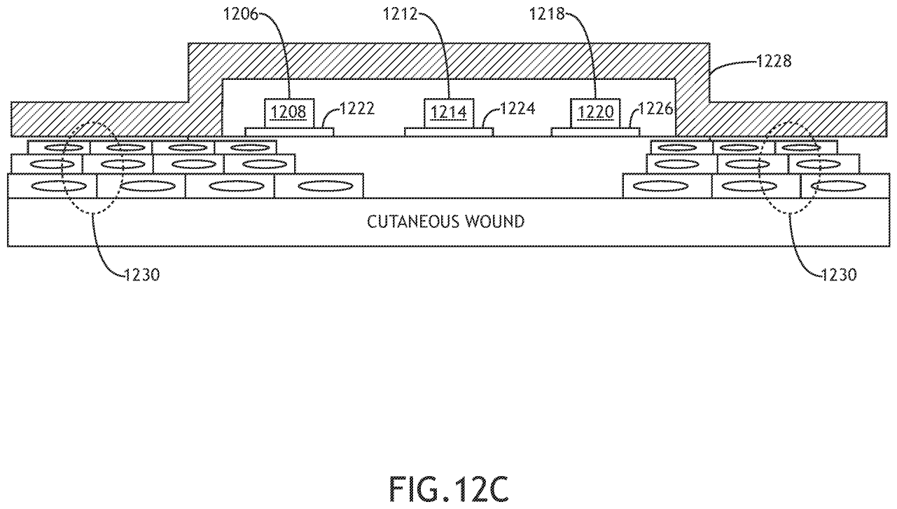

FIGS. 12A through 12C show a HIC-based electrical stimulation device 1200, in accordance with one or more embodiments of this disclosure. In embodiments, the HIC-based electrical stimulation device 1200 includes a substrate 1201 (e.g., a flexible or rigid polymer substrate) configured to overlay a cutaneous wound (e.g., as illustrated in FIG. 12B). In some embodiments, the substrate 1201 includes a transparent window/cutout 1203 for testing or viewing healing activity in the underlying cutaneous wound.

The substrate 1201 may have a plurality of channels (e.g., channels 1206, 1212, 1218) embedded within or attached to the substrate 1201, with each of the channels containing a salt solution (e.g., salt solution 1208, 1214, 1220) and being at least partially bound by a hydrogel membrane (e.g., hydrogel membrane 1222, 1224, 1226) that defines a barrier between the salt solution and the cutaneous wound. As shown in FIG. 12A, the HIC channels (e.g., channels 1206, 1212, 1218) may be coupled to a power source 1202 (e.g., DC power supply) by respective electrodes (e.g., electrodes 1204, 1210, 1216). In embodiments, the salt solution may be a phosphate or sulfate salt solution and the hydrogel membrane may comprise PEG hydrogel matrix (e.g., as described with regard to systems/devices in FIGS. 1 through 11B). In this regard, the hydrogel membrane is ionically conductive configured to transmit the ion current to the cutaneous wound while also being configured to configured to contain salt ions stably within the channel due to aqueous two-phase separation (ATPS).

In an embodiment, the substrate 1201 includes channel 1206 coupled to electrode 1204 and channel 1212 coupled to electrode 1210. Electrode 1204 is configured to apply an electrical current to channel 1206 to induce an ion current in salt solution 1208, wherein the ion current acts on the cutaneous wound to stimulate healing. For example, the ion current may flow through a portion of the cutaneous wound from the channel 1206 to channel 1212, wherein channel 1212 is coupled to electrode 1210 (a counter electrode) to complete the circuit between channels 1206 and 1212.

In some embodiments, the substrate 1201 may only include two HIC channels (e.g., only channels 1206 and 1212). In other embodiments, such as the embodiments illustrated in FIGS. 12A through 12C, the substrate 1201 includes at least one additional channel (channel 1218) that is also coupled to a working electrode (electrode 1216). Electrode 1216 may be configured to apply an electrical current to channel 1218 to induce a second ion current in salt solution 1220. This second ion current may also act on the cutaneous wound to stimulate healing. For example, the second ion current may flow through a portion of the cutaneous wound from the channel 1218 to channel 1212. In some embodiments, the electrode polarities may be reversed such that ion currents flow from electrode 1212 to electrodes 1206 and 1218. Furthermore, the substrate 1201 may include any number of HIC channels with alternating electrode polarity from one channel to the next.

As shown in FIG. 12C, the HIC-based electrical stimulation device 1200 may further include a splint 1228 configured to hold the substrate 1201 against the cutaneous wound. In some embodiments, the splint 1228 and/or substrate 1201 may be held in place by two or more sutures 1230.

The HIC-based electrical stimulation device 1200 is configured to convert electron current to ion current at the current source/HIC interface through EC reactions. In embodiments, the high-concentration salt solutions (e.g., phosphate, sulfate, citrate, carbonate, or polyacrylate salt solution) in the HIC-based electrical stimulation device 1200 have high efficiency in buffering the pH changes. They can also absorb the heat generated by the EC reactions. As a result, the pH and temperature changes do not affect biological tissue at the site of the cutaneous wound. This allows for prolonged electrical stimulation at sufficiently high current intensity to improve the rate of wound healing.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

1. An iontophoresis device, comprising:
a first chamber containing a salt solution;
a second chamber containing a therapeutic solution, the second chamber being configured to interface with a portion of a surface overlaying a target region;
a hydrogel membrane separating the first chamber from the second chamber; and
an electrode configured to apply an electrical current to the first chamber to induce an ion current in the salt solution, wherein the ion current acts on the second chamber to iontophoretically transport molecules from the therapeutic solution across the surface to the target region.

2. The iontophoresis device of claim 1, wherein the hydrogel membrane is ionically conductive configured to transmit the ion current to the second chamber.

3. The iontophoresis device of claim 1, wherein the hydrogel membrane comprises a polyethylene glycol (PEG) hydrogel matrix.

4. The iontophoresis device of claim 1, wherein the hydrogel membrane is configured to configured to contain salt ions stably within the first chamber due to aqueous two-phase separation (ATPS).

5. The iontophoresis device of claim 1, wherein the salt solution comprises at least one of: a phosphate salt solution, a sulfate salt solution, citrate salt solution, carbonate salt solution, or polyacrylate salt solution.

6. The iontophoresis device of claim 1, wherein the salt solution contains an amount of salt required to maintain a pH in the range of 6.5 to 8.5 at the surface overlaying the target region.

7. The iontophoresis device of claim 1, wherein the salt solution is configured to absorb heat generated by electrode overpotential to maintain a temperature below 43° C. at the surface overlaying the target region.

8. The iontophoresis device of claim 1, wherein the first chamber, the second chamber, and the hydrogel membrane are embedded within an ocular lens.

9. The iontophoresis device of claim 7, wherein the ocular lens is formed from a hydrogel.

10. The iontophoresis device of claim 8, wherein the ocular lens is formed from the same hydrogel as the hydrogel membrane.

11. The iontophoresis device of claim 9, wherein the hydrogel membrane is part of the ocular lens.

12.-19. (canceled)

20. A hydrogel ionic circuit (HIC) electrode, comprising:
a chamber containing a salt solution;
an electrode configured to apply an electrical current to the chamber to induce an ion current in the salt solution; and
a hydrogel membrane defining a barrier for the salt solution, wherein the hydrogel membrane is ionically conductive and configured to transmit the ion current.

21. The HIC electrode of claim 20, wherein the hydrogel membrane comprises a polyethylene glycol (PEG) hydrogel matrix.

22. The HIC electrode of claim 20, wherein the hydrogel membrane is configured to configured to contain salt ions stably within the chamber due to aqueous two-phase separation (ATPS).

23. The HIC electrode of claim 20, wherein the salt solution comprises at least one of: a phosphate salt solution, a sulfate salt solution, citrate salt solution, carbonate salt solution, or polyacrylate salt solution.

24. A method of delivering a therapeutic agent across an ocular surface, the method comprising:
disposing a salt solution within a first chamber;
disposing a therapeutic solution containing the therapeutic agent within a second chamber, wherein the first chamber and the second chamber are separated by a hydrogel membrane;
interfacing the second chamber with the ocular surface; and
applying an electrical current to the first chamber to induce an ion current in the salt solution, wherein the ion current acts on the second chamber to iontophoretically transport the therapeutic agent from the therapeutic solution across the ocular surface.

25. (canceled)
